(19) United States
(12) Patent Application Publication
(10) Pub. No.: US 2022/0272953 A1
(43) Pub. Date: Sep. 1, 2022

(54) GENETICALLY MODIFIED NON-HUMAN ANIMAL WITH HUMAN OR CHIMERIC IL33

(71) Applicant: Biocytogen Pharmaceuticals (Beijing) Co., Ltd., Beijing (CN)

(72) Inventors: Yuelei Shen, Beijing (CN); Yanan Guo, Beijing (CN); Yang Bai, Beijing (CN); Chengzhang Shang, Beijing (CN); Rui Huang, Beijing (CN); Meiling Zhang, Beijing (CN); Jiawei Yao, Beijing (CN); Chaoshe Guo, Beijing (CN)

(21) Appl. No.: 17/631,048

(22) PCT Filed: Jul. 29, 2020
(86) PCT No.: PCT/CN2020/105529

§ 371 Date: Jan. 28, 2022

(30) Foreign Application Priority Data
Jul. 29, 2019 (CN) 201910688648.5

(51) Int. Cl. A01K 67/027; C07K 14/54

(52) U.S. Cl. CPC A01K 67/0278; C07K 14/54; A01K 2217/077; A01K 2227/105; A01K 2267/0387; A01K 2267/0368; A01K 2267/0331

(57) ABSTRACT

Provided are genetically modified non-human animals that express a human or chimeric (e.g., humanized) IL33, and methods of use thereof.

### CLAIM OF PRIORITY

This application claims the benefit of Chinese Patent Application App. No. 201910688648.5, filed on Jul. 29, 2019. The entire contents of the foregoing are incorporated herein by reference.

### TECHNICAL FIELD

This disclosure relates to genetically modified animal expressing human or thimeric (e.g., humanized) IL33, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost tier drug research and development.

### SUMMARY

This disclosure is related to an animal model with human IL33 or chimeric IL33. The animal model can express human IL33 or chimeric IL33 (e.g., humanized IL33) protein in its body. It can be used in the studies on the function of IL33 gene, and can be used in the screening and evaluation of anti-human IL33 antibodies. In addition, the animal models prepared by the methods described herein can be used in drug screening, pharmacodynamics studies, treatments for immune-related diseases (e.g., allergic disorders), and cancer therapy for human IL33 target sites; they can also be used to facilitate the development and design of new drugs, and save time and cost. In summary, this disclosure provides a powerful tool for studying the function of IL33 protein and a platform for screening drugs, e.g., antibodies, against allergic disorders (e.g., asthma).

In one aspect, the disclosure is related to a genetically-modified, non-human animal whose genome comprises at least one chromosome comprising a sequence encoding a human or chimeric IL33.

In some embodiments, the sequence encoding the human or chimeric IL33 is operably linked to an endogenous regulatory element at the endogenous IL33 gene locus in the at least one chromosome.

In some embodiments, the sequence encoding a human or chimeric IL33 is operably linked to an endogenous 5′ untranslated region (5′-UTR).

In some embodiments, the sequence encoding a human or chimeric IL33 comprises a sequence encoding an amino acid sequence that is at least 70%, 75%, 80%, 85%, 90%, 95%, 99%, or 100% identical to human IL33 (SEQ ID NO: 8, SEQ ID NO: 9, SEQ ID NO: 10, SEQ ID NO: 11, or SEQ ID NO: 12).

In some embodiments, the sequence encoding a human or chimeric IL33 comprises a sequence encoding an amino acid sequence that is at least 70%, 75%, 80%, 85%, 90%, 95%, 99%, or 100% identical to SEQ ID NO: 8.

In some embodiments, the sequence encoding a human or chimeric IL33 comprises a sequence encoding an amino acid sequence that is at least 70%, 75%, 80%, 85%, 90%, 95%, 99%, or 100% identical to SEQ ID NO: 9.

In some embodiments, the sequence encoding a human or chimeric IL33 comprises a sequence encoding an amino acid sequence that is at least 70%, 75%, 80%, 85%, 90%, 95%, 99%, or 100% identical to SEQ ID NO: 10.

In some embodiments, the sequence encoding a human or chimeric IL33 comprises a sequence encoding an amino acid sequence that is at least 70%, 75%, 80%, 85%, 90%, 95%, 99%, or 100% identical to SEQ ID NO: 11.

In some embodiments, the sequence encoding a human or chimeric IL33 comprises a sequence encoding an amino acid sequence that is at least 70%, 75%, 80%, 85%, 90%, 95%, 99%, or 100% identical to SEQ ID NO: 12.

In some embodiments, the animal is a mammal, e.g., a monkey, a rodent, or a mouse. In some embodiments, the mammal is a mouse.

In some embodiments, the animal does not express endogenous IL33.

In some embodiments, the animal has one or more cells expressing human or chimeric IL33.

In some embodiments, the expressed human or chimeric IL33 can bind to human interleukin 1 receptor-like 1 (IL1RL1). In some embodiments, the expressed human or chimeric IL33 can bind to endogenous IL1RL1.

In one aspect, the disclosure is related to a genetically-modified, non-human animal. In some embodiments, the genome of the animal comprises a replacement of a sequence encoding a region of endogenous IL33 with a sequence encoding a corresponding region of human IL33 at an endogenous IL33 gene locus.

In some embodiments, the sequence encoding the corresponding region of human IL33 is operably linked to an endogenous regulatory element at the endogenous IL33 locus.

In some embodiments, the animal does not express endogenous IL33, and the animal has one or more cells expressing human or chimeric IL33.

In some embodiments, the animal is a mouse, and the sequence encoding the corresponding region of human IL33 comprises exon 2, exon 3, exon 4, exon 5, exon 6, exon 7 and/or exon 8, or a part thereof, of human IL33 gene.

In some embodiments, the animal is heterozygous with respect to the replacement at the endogenous IL33 gene locus. In some embodiments, the animal is homozygous with respect to the replacement at the endogenous IL33 gene locus.

In one aspect, the disclosure is related to a method for making a genetically-modified, non-human animal, comprising: replacing in at least one cell of the animal, at an endogenous IL33 gene locus, a sequence encoding a region of an endogenous IL33 with a sequence encoding a corresponding region of human IL33.

In some embodiments, the sequence encoding the corresponding region of human IL33 comprises exon 2, exon 3, exon 4, exon 5, exon 6, exon 7, and/or exon 8, or a part thereof, of a human IL33 gene.

In some embodiments, the sequence encoding the corresponding region of human IL33 encodes an amino acid sequence that is at least 70%, 75%, 80%, 85%, 90%, 95%, 99%, or 100% identical to SEQ ID NO: 8, SEQ ID NO: 9, SEQ ID NO: 10, SEQ ID NO: 11, or SEQ ID NO: 12.

In some embodiments, the animal is a mouse, and the endogenous IL33 locus comprises exon 2, exon 3, exon 4, exon 5, exon 6, exon 7, and/or exon 8, or a part thereof, of the mouse IL33 gene.

In one aspect, the disclosure is related to a non-human animal comprising at least one cell comprising a nucleotide sequence encoding an exogenous IL33 polypeptide. In some embodiments, the exogenous IL33 polypeptide comprises at least 50 contiguous amino acid residues that are identical to the corresponding contiguous amino acid sequence of a human IL33. In some embodiments, the animal expresses the exogenous IL33.

In some embodiments, the exogenous IL33 polypeptide comprises an amino acid sequence that is at least 90%, 95%, or 99% identical to SEQ ID NO: 8, SEQ ID NO: 9, SEQ ID NO: 10, SEQ ID NO: 11, or SEQ ID NO: 12.

In some embodiments, the nucleotide sequence is operably linked to an endogenous IL33 regulatory element of the animal.

In some embodiments, the nucleotide sequence is integrated to an endogenous IL33 gene locus of the animal.

In some embodiments, the animal in its genome comprises from 5′ to 3″: a mouse 5′ UTR, a sequence encoding the exogenous IL33 polypeptide, and a mouse 3′ UTR.

In one aspect, the disclosure is related to a method of making a genetically-modified mouse cell that expresses a chimeric IL33, the method comprising: replacing at an endogenous mouse IL33 gene locus, a nucleotide sequence encoding a region of mouse IL33 with a nucleotide sequence encoding a corresponding region of human IL33, thereby generating a genetically-modified mouse cell that includes a nucleotide sequence that encodes the chimeric IL33. In some embodiments, the mouse cell expresses the chimeric IL33.

In some embodiments, the nucleotide sequence encoding the chimeric IL33 is operably linked to an endogenous IL33 regulatory region, e.g., promoter.

In some embodiments, the animal further comprises a sequence encoding an additional human or chimeric protein. In some embodiments, the additional human or chimeric protein is programmed cell death protein I (PD-1), cytotoxic T-lymphocyte-associated protein 4 (CTLA-4), Lymphocyte Activating 3 (LAG-3), IL 15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40). In some embodiments, the additional human or chimeric protein is PD-1.

In some embodiments, the animal or mouse further comprises a sequence encoding an additional human or chimeric protein. In some embodiments, the additional human or chimeric protein is PD-1, CTLA-4, LAG-3,11,15 receptor, BTLA, PD-L1, CD3, CD27, CD28, CD47, CD137, CD154, TIGIT, TIM-3, GITR, SIRPα or OX40. In some embodiments, the additional human or chimeric protein is PD-1.

In one aspect, the disclosure is related to a method of determining effectiveness of an anti-IL33 antibody for treating an allergic disorder, comprising: a) administering the anti-IL33 antibody to the animal as described herein; in some embodiments, the animal has the allergic disorder; and b) determining effects of the anti-IL33 antibody in treating the allergic disorder.

In some embodiments, the allergic disorder is asthma. In some embodiments, the animal is a mouse and the asthma is induced by treating the mouse with ovalbumin and aluminum hydroxide.

In some embodiments, the effects are evaluated by serum IgE levels; pathological lung histology features; number of leukocytes (CD45+ cells), eosinophils (Eos), or neutrophils in bronchoalveolar lavage fluid (BALF); or percentages of eosinophils or neutrophils cells in CD45+ cells in bronchoalveolar lavage fluid (BALF).

In some embodiments, the allergic disorder is hay fever.

In one aspect, the disclosure is related to a method of determining effectiveness of an anti-IL33 antibody for reducing an inflammation, comprising: a) administering the anti-IL33 antibody to the animal as described herein; in some embodiments, the animal has the inflammation; and b) determining effects of the anti-IL33 antibody for reducing the inflammation.

In one aspect, the disclosure is related to a method of determining effectiveness of an anti-IL33 antibody for treating an autoimmune disorder, comprising: a) administering the anti-IL33 antibody to the animal as described herein; in some embodiments, the animal has the autoimmune disorder; and b) determining effects of the anti-IL33 antibody for treating the auto-immune disease.

In one aspect, the disclosure is related to a method of determining effectiveness of an anti-IL33 antibody for treating a cancer, comprising: a) administering the anti-IL33 antibody to the animal as described herein; in some embodiments, the animal has the cancer; and b) determining inhibitory effects of the anti-IL33 antibody for treating the cancer.

In some embodiments, the cancer is a tumor, and determining the inhibitory effects of the treatment involves measuring the tumor volume in the animal.

In some embodiments, the cancer is breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, or bone cancer.

In one aspect, the disclosure is related to a method of determining toxicity of an anti-IL33 antibody, the method comprising a) administering the anti-IL33 antibody to the animal as described herein; and b) determining weight change of the animal. in some embodiments, the method further comprising performing a blood test (e.g., determining red blood cell count).

In one aspect, the disclosure is related to a protein comprising an amino acid sequence. In some embodiments, the amino acid sequence is one of the following:
(a) an amino acid sequence set forth in SEQ ID NOS: 8-12;
(b) an amino acid sequence that is at least 90% identical to SEQ ID NOS: 8-12;
(c) an amino acid sequence that is at least 91%, 92%, 93%, 94%, 95%, 96%, 97%, 98%, or 99% identical to SEQ ID NOS: 8-12;
(d) an amino acid sequence that is different from the amino acid sequence set forth in SEQ ID NOS: 8-12 by no more than 10, 9, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NOS: 8-12.

In one aspect, the disclosure is related to a nucleic acid comprising a nucleotide sequence. In some embodiments, the nucleotide sequence is one of the following:
(a) a sequence that encodes the protein as described herein;
(b) SEQ ID NO: 7;
(c) a sequence that at least 90% identical to SEQ ID NO: 7; and
(d) a sequence that is at least 91%, 92%, 93%, 94%, 95%, 96%, 97%, 98%, or 99% identical to SEQ ID NO: 7.

In one aspect, the disclosure is related to a cell comprising the protein as described herein and/or the nucleic acid as described herein. In one aspect, the disclosure is related to an animal comprising the protein as described herein and/or the nucleic acid as described herein.

The disclosure further relates to a IL33 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the IL33 gene function, human IL33 antibodies, the drugs or efficacies for human IL33 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse IL33 gene locus.

FIG. 1B is a schematic diagram showing human IL33 gene locus.

FIG. 2 is a schematic diagram showing humanized IL33 gene locus.

FIG. 3 is a schematic diagram showing an IL33 gene targeting strategy.

FIG. 4 shows Southern. Blot results. WT is wild-type.

FIG. 5A shows PCR identification result of samples collected from tails of F1 generation mice. Primers IL33-WT-F/IL33-WT-R were used for amplification. WT is wild-type, H₂O is a blank control, + is positive control and M is marker.

FIG. 5B shows PCR identification result of samples collected from tails of F1 generation mice. Primers IL33-WT-F/IL33-Mut-R were used for amplification. WT is wild-type, H₂O is a blank control, + is positive control and M is marker.

FIG. 5C shows PCR identification result of samples collected from tails of F1 generation mice. Primers IL33-Frt-F/IL33-Frt-R were used for amplification. WT is wild-type, H₂O is a blank control, + is positive control and M is marker.

FIG. 5C shows PCR identification result of samples collected from tails of F1 generation mice. Primers IL33-Flp-F2/IL33-Flp-R2 were used for amplification. WT is wild-type, H₂O is a blank control, + is positive control and M is marker.

FIG. 6A shows mouse IL33 protein expression level measured by ELISA. +/+ represents wild-type C57BL/6 mice. H/+ represents IL33 gene humanized heterozygous mice.

FIG. 6B shows human IL33 protein expression level measured by ELISA. +/+ represents wild-type C57BL/6 mice. H/+ represents IL33 gene humanized heterozygous mice.

FIG. 7 shows an experimental protocol of using IL33 gene humanized mice to make an inducible asthma model and to assess treatment efficacy of anti-human IL33 antibody Etokimab. The IL33 gene humanized mice were induced by ovalbumin and aluminum hydroxide at day 0, day 7 and day 14.

FIG. 8 is a graph showing total umber of leukocytes (mouse CD45+ cells, or mCD45+ cells) in bronchoalveolar lavage fluid (BALF) of IL33 gene humanized mice induced by ovalbumin combined with aluminum hydroxide or PBS (control group G10, wherein OVA-induced group G1 had more leukocytes than the PBS control group G1 and the anti-IL33 antibody-treated groups (G3 and G4).

FIG. 9 is a graph showing total number of eosinophils (Eos) cells in bronchoalveolar lavage fluid (BALF) of IL33 gene humanized mice induced by ovalbumin combined with aluminum hydroxide or PBS (control group G1), wherein OVA-induced group G2 had more Eos cells than the PBS control group G1 and Etokimab-treated groups (G3 and G4).

FIG. 10 is a graph showing the proportion of neutrophil cells (Neu %) in bronchoalveolar lavage fluid (BALF) of IL33 humanized mice induced by ovalbumin combined with aluminum hydroxide (OVA) or PBS (control group). The result shows mice in OVA-induced group had significantly more neutrophils compared with the PBS control group G1 and the Etokimab-treated groups (G3 and G4).

FIG. 11 is a graph showing serum IgE levels of IL33 humanized mice induced by ovalbumin combined with aluminum hydroxide. The OVA-induced group (G2) had high serum IgE levels than the control group G1 and Etokimab-treated group G4.

FIG. 12 is a set of graphs showing airway tissue section H&E staining result of IL33 gene humanized mice induced by ovalbumin combined with aluminum hydroxide in Example 3. The airway of the control group (G1) mice showed no inflammation while the OVA-induced group (G2) had peribronchial and perivascular inflammation and increased mucus secretion levels. The mice in the treatment group (G3, G4) had decreased inflammatory infiltration and mucus secretion as compared to the G2 group.

FIG. 13 the alignment between mouse IL33 amino acid sequence (NP_59 8536.2; SEQ ID NO: 2) and human IL33 amino acid sequence (NP_254274.1; SEQ ID NO: 8).

### DETAILED DESCRIPTION

This disclosure relates to transgenic non-human animal with human or chimeric humanized) IL33, and methods of use thereof.

Interleukin 33 (IL-33) is a protein that in humans is encoded by the IL33 gene. IL-33 is a member of the IL-1 family that potently drives production of T helper-2 (Th2)-associated cytokines (e.g., IL-4). IL33 is a ligand for ST2 (IL1RL1), an IL-1 family receptor that is highly expressed on Th2 cells, mast cells and group 2 innate lymphocytes. IL-33 is expressed by a wide variety of cell types, including fibroblasts, mast cells, dendritic cells, macrophages, osteoblasts, endothelial cells, and epithelial cells.

The full-length IL-33 contains 270 amino acids in human and 266 in mice, which harbors a homeodomain-like helix-turn-helix domain presumably allowing to bind to DNA. The release of IL-33 can be associated with mechanical and oxidative stress, necrotic cell death, or cell activation through ATP signaling in the absence of cell death. IL-33 is rapidly released from cells during necrosis or tissue injury, and signals through a cell surface receptor complex, ST2 (IL-1 receptor-like 1, IL1RL1) and IL1RAcP (IL-1 receptor accessory protein), to initiate inflammatory pathways in immune cells, such as type-2 innate lymphoid cells (ILC2), mast cells and natural killer (NK) cells. Thus, IL33 antibodies can be potentially used to treat allergic disorders (e.g., asthma) or inflammatory diseases.

Experimental animal models are an indispensable research tool for studying the effects of these antibodies (e.g., IL33 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

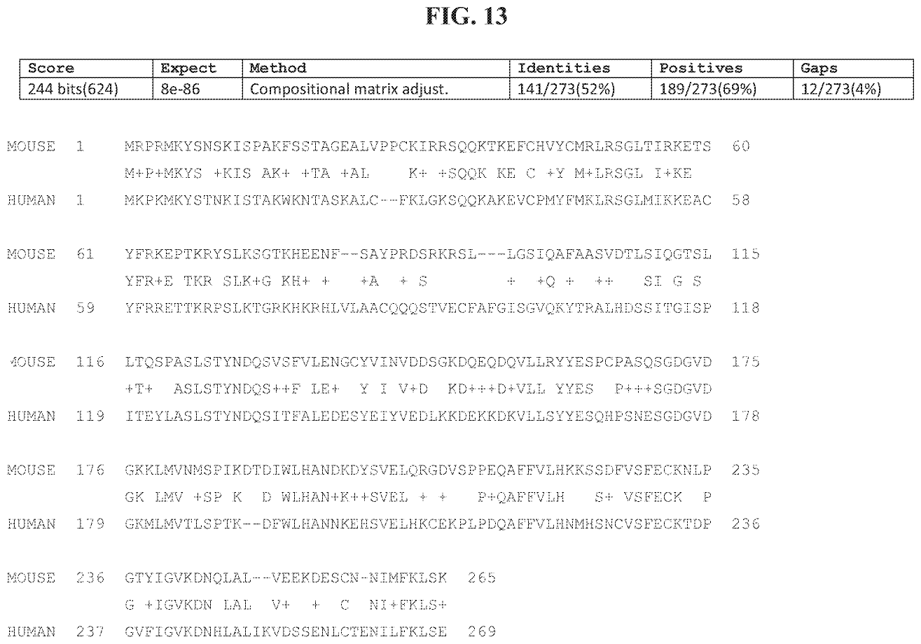

Particularly, the present disclosure demonstrates that a replacement with human IL-33 sequence at an endogenous IL-33 locus under control of endogenous regulatory elements provides a physiologically appropriate expression pattern and level that results in a useful humanized animal. As shown in the present disclosure, while the human IL33 sequence is quite different from the animal IL33 sequence (see e.g., FIG. 13), the human IL33 gene sequences are properly spliced in the animal, and the expressed human IL33 is functional and can properly interact with the endogenous IL33 receptor. Both genetically modified animals that are heterozygous or homozygous for humanized IL33 are grossly normal and can be used to evaluate the efficacy of anti-human IL-33 antibodies in an immune disorder model.

Unless otherwise specified, the practice of the methods described herein can take advantage of the techniques of cell biology, cell culture, molecular biology, transgenic biology, microbiology, recombinant DNA and immunology.

### IL33

Interleukin 33 (IL-33 or IL33) was identified as a member of the IL-1 family of cytokines and the ligand for ST2L. It is constitutively expressed in many tissues and by a wide variety of cells. It is also induced in response to various stimuli in epithelial cells, myofibroblasts, adipocytes, endothelial cells, smooth muscle cells, and macrophages predominantly as a pro-inflammatory cytokine. IL-33 is about 30 kDa that functions dually as a transcription factor and a cytokine. Its N-terminus contains a nuclear localization signal, a DNA-binding homeodomain-like helix-turn-helix motif, and a chromatin binding domain, while the C-terminus contains an IL-1 like cytokine domain.

Full length IL-33 is targeted to the nucleus upon synthesis, where it binds to chromatin and is thought to regulate gene expression by a number of mechanisms. It can bind to histones H2A and H2B and can activate histone deacetylase-3 (HDAC) activity thereby affecting gene expression by remodeling chromatin structure and by epigenetic mechanisms. It has been shown to interact with the N-terminal domain of the p65 subunit of nuclear factor κB (NF-κB) to repress the expression of NF-κB-regulated genes that are necessary for pro-inflammatory signaling. In response to cellular damage, tissue injury or viral infection, IL-33 is quickly released from the nucleus of necrotic cells and secreted into extracellular space where it can bind to the membrane-bound. ST2L receptor through its cytokine domain. Binding to its receptor triggers an inflammatory cascade, thus, IL-33 acts as an “alarmin” and is considered a damage-associated molecular pattern (DAMP). The nuclear and cytokine functions of IL-33 are tightly regulated through its localization. Full length nuclear IL-33 acts as a transcription factor that modulates cytokine gene expression and its nuclear compartmentalization is a deterrent to unleashing damaging inflammation instigated by its alarmin and cytokine functions.

Full-length (FL) IL-33 can function as a cytokine and can be degraded by the pro-apoptotic caspases 3 and 7, resulting in its inactivation. Thus, rather than a pre-requisite for its activation, cleavage by these caspases is thought to act as a switch to extinguish the pro-inflammatory activity of IL-33, ensuring immune tolerance during apoptosis by preventing its secretion. On the other hand, under inflammatory conditions, full length IL-33 are cleaved by the serine proteases cathepsin G and elastase released by neutrophils to generate mature forms that are ten-fold more bioactive than FL-IL-33. IL-33 can also be cleaved by chymase and tryptase proteases secreted by activated mast cells, critical effector cells in allergic disorders, to potently activate group 2 innate lymphoid cells. Cleavage by mast cell proteases generate three different mature isoforms of IL-33 that are 30-fold more bioactive than FL-IL-33. it is important to note that both mast cells and neutrophils are abundantly recruited into the TME (tumor microenvironment). The mature forms of IL-33 lack the N-terminal domain and function as IL-1-like cytokines through their C-terminal domain. Thus, the activity of IL-33 can be amplified in the context of an inflammatory microenvironment through the action of proteases secreted by innate cells that are recruited in response to injury or inflammation.

### The IL-33/ST2 Pathway

The ST2 receptor had been extensively studied prior to the discovery of its ligand IL-33. Suppression of tumorigenicity 2 (ST2) was first identified in murine fibroblasts as an oncogene-induced gene. It is encoded by IL1RL1 (IL-1 receptor-like 1) that produces four isoforms through alternative splicing: ST2L (ligand), sST2, ST2V (variant), and ST2LV (ligand variant). ST2L is a membrane embedded receptor that is highly homologous to IL-I type-1 receptors and harbors three Ig-like extracellular domains, a transmembrane spanning region, and an ILI-R1-like intracellular domain. ST2L forms a heterodimeric transmembrane receptor complex with the IL1-receptor accessory protein, IL1-RAcP that is necessary for signal transduction upon binding of IL-33.

As a nuclear factor, IL-33 binds to chromatin to repress the expression of inflammatory responses. As a cytokine, IL-33 is secreted into extracellular space in response to cell damage or mechanical injury. IL-33 can then bind to the ST2L receptor, via its C-terminal IL-1 like cytokine domain, inducing a conformational change that results in recruitment of IL-1RAcP (IL-1 receptor accessory protein) to form a heterodimeric receptor complex on the cell membrane. Hetero-dimerization brings together the intracellular domains of the two transmembrane proteins, and its assembly initiates the recruitment of adaptor molecules through which the IL-33 signal is transduced.

A detailed description of IL33 and its function can be found, e.g., in Larsen et al., “The role of IL-33/ST2 pathway in tumorigenesis.” International Journal of Molecular Sciences 19.9 (2018): 2676; Shen et al., “Interleukin-33 in malignancies: friends or foes?.” Frontiers in Immunology 9 (2018):3051; each of which is incorporated by reference in its entirety.

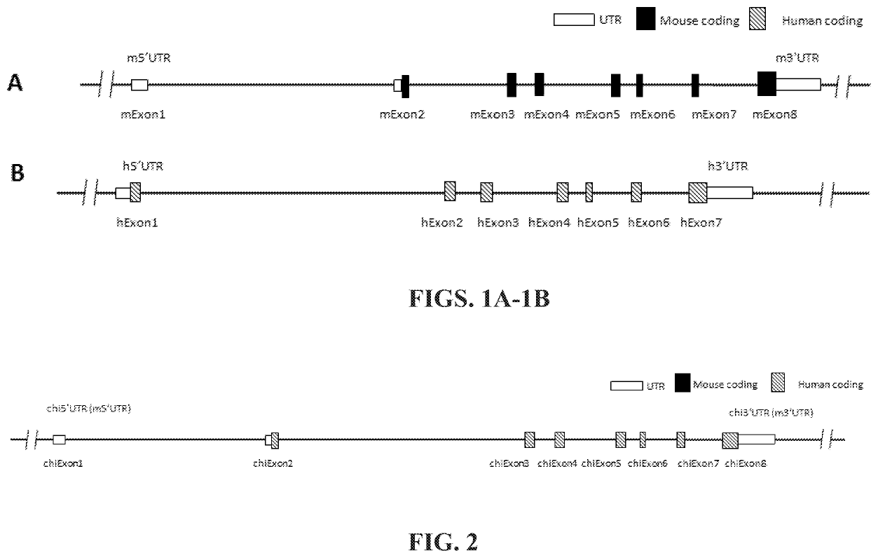

In human genomes, IL33 gene (Gene ID: 90865) locus has 8 exons, exon 1, exon 2, exon 3, exon 4, exon 5, exon 6, exon 7, and exon 8 (FIG. 1A). The nucleotide sequence for human IL33 mRNA is NM_033439.3 (SEQ ID NO: 3), and the amino acid sequence for human IL33 is NP_254274.1 (SEQ ID NO: 8). The location for each exon and each region in human IL33 nucleotide sequence and amino acid sequence is listed below:

TABLE 1

| | NM_033439.3 | NP_254274.1 |
|---|---|---|
| Human IL33 | 2718 bp | 270 amino acids |
| (approximate location) | (SEQ ID NO: 3) | (SEQ ID NO: 8) |
| Exon 1 | 1-67 | Non-coding |
| Exon 2 | 68-169 | 1-30 |
| Exon 3 | 170-295 | 31-72 |
| Exon 4 | 296-421 | 73-114 |
| Exon 5 | 422-547 | 115-156 |
| Exon 6 | 548-598 | 157-173 |
| Exon 7 | 599-690 | 174-204 |
| Exon 8 | 691-2706 | 205-270 |
| Donnor region in Example | 79-891 | 1-270 |

In mice, IL33 gene locus has 8 exons, exon 1, exon 2, exon 3, exon 4, exon 5, exon 6, exon 7, and exon 8 (FIG. 1B). The nucleotide sequence for mouse IL33 mRNA is NM_133775.3 (SEQ ID NO: 1), the amino acid sequence for mouse IL33 is NP_598536.2 (SEQ ID NO: 2). The location for each exon and each region in the mouse IL33 nucleotide sequence and amino acid sequence is listed below:

TABLE 2

| | NM_133775.3 | NP_598536.2 |
|---|---|---|
| Mouse IL33 | 2538 bp | 266 amino acids |
| (approximate location) | (SEQ ID NO: 1) | (SEQ ID NO: 2) |
| Exon 1 | 1-49 | Non-coding |
| Exon 2 | 50 . . . 157 | 1-32 |
| Exon 3 | 158 . . . 283 | 33-74 |
| Exon 4 | 284 . . . 394 | 75-111 |
| Exon 5 | 395 . . . 520 | 112-153 |
| Exon 6 | 521 . . . 571 | 154-170 |
| Exon 7 | 572 . . . 669 | 171-203 |
| Exon 8 | 670 . . . 2538 | 204-266 |
| Replaced region in Example | 61-861 | 1-266 |

The mouse IL33 gene (Gene ID: 77125) is located in Chromosome 19 of the mouse genome, which is located from 29925114 to 29960715, of NC_000085.6 (GRCm38.p4 (GCF_000001.635.24)). The 5′-UTR is from 29,925,114 to29,925,161 and 29,949,660 to 29,949,670, exon 1 is from 29,925,114 to 29,925,161, the first intron is from 29.925,162 to 29,949,659, exon 2 is from 29,949,660 to 29.949,767, the second intron is from 29,949,768 to 29,951,975, exon 3 is from 29,951,976 to 29,952,101, the third intron is from 29,952,102 to 29,952,729, exon 4 is from 29,952,730 to 29,952,840, the fourth intron is from 29,952,841 to 29,954,542, exon 5 is from 29,954,543 to 29,954,668, the fifth intron is from 29,954,669 to 29,955,200, the exon 6 is from 29,955,201 to 29,955,251, the sixth intron is from 29,955,252 to 29,956,900, the exon 7 is from 29,956,901 to 29,956,998 the seventh intron is from 29,956,999 to 29,958,849, the exon 8 is from 29,958,850 to 29,960,718, and the 3′-UTR is from 29,959,042 to 29,960,718, based on transcript NM_1133775.3. All relevant information for mouse IL33 locus can be found in the NCBI website with Gene ID: 77125, which is incorporated by reference herein in its entirety.

FIG. 13 shows the alignment between mouse IL33 amino acid sequence (NP_598536.2; SEQ ID NO: 2) and human IL33 amino acid sequence (NP_254274.1; SEQ ID NO: 8). Thus, the corresponding amino acid residue or region between human and mouse IL33 can be found in FIG. 13.

IL33 genes, proteins, and locus of the other species are also known in the art. For example, the gene ID for IL33 in Rattus norvegicus (rat) is 361749, the gene ID for IL33 in Macaca mulatta (Rhesus monkey) is 717301, the gene IL) for IL33 in Sus scrofa (pig) is 100518643, the gene ID for IL33 in Oryctolagus cuniculus (rabbit) is 100356081, and the gene ID for IL33 in Felis catus domestic cat) is 101093403. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) IL33 nucleotide sequence and/or amino acid sequences. In some embodiments, the entire sequence of mouse exon 1, exon 2, exon 3, exon 4, exon 5, exon 6, exon 7, exon 8, and/or signal peptide, are replaced by the corresponding human sequence. In some embodiments, a “region” or a “portion” of mouse exon 1, exon 2, exon 3, exon 4, exon 5, exon 6, exon 7, exon 8, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, or signal peptide. In some embodiments, a region, a portion, or the entire sequence of mouse exon 1 exon 2, exon 3, exon 4, exon 5, exon 6, exon 7, and/or exon 8 (e.g., a portion of exon 2, exon 3, exon 4, exon 5, exon 6, exon 7, and a portion of exon 8 of mouse IL33 gene) are replaced by human exon 1, exon 2, exon 3, exon 4, exon 5, exon 6, exon 7, and/or exon 8 (e.g., a portion of exon 2, exon 3, exon 4, exon 5, exon 6, exon 7, and a portion of exon 8 of human IL33 gene) sequence.

In some embodiments, the present disclosure also provides a chimeric (e.g., humanized) or human IL33 nucleotide sequence and/or amino acid sequences, wherein in some embodiments, at least 1%, 2%, 1%, 4%, 5%, 6%, 7%, 8%, 9%, 10%, 1S%, 20%, 25%, 30%, 35%, 40%, 4S%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL33 mRNA sequence (e.g., SEQ ID NO: 1), mouse IL33 amino acid sequence (e.g., SEQ ID NO: 2), or a portion thereof (e.g., exon 1, a portion of exon 2., and a portion of exon 8, of NM_133775.3 (SEQ ID NO: 1)); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL33 mRNA sequence (e.g., SEQ ID NO: 3), human IL33 amino acid sequence SEQ ID NOS: 8-12), or a portion thereof (e.g., a portion of exon 2, exon 3, exon 4, exon 5, exon 6, exon 7, and a portion of exon 8, of NM_033439.3 (SEQ ID NO: 3)).

In some embodiments, the sequence encoding amino acids 1-266 of mouse IL33 (SEQ ID NO: 2) is replaced. In some embodiments, the sequence is replaced by a sequence encoding a corresponding region of human IL33 (e.g., amino acids 1-270 of human IL33 (SEQ ID NO: 4)).

In some embodiments, the nucleic acid sequence described herein are operably linked to a promotor or regulatory element, e.g., an endogenous mouse IL33 promotor, an inducible promoter, an enhancer, and/or mouse or human regulatory elements. In some embodiments, the nucleic acid sequence described herein is connected to an endogenous 5′ UTR. In some embodiments, the 5′UTR is identical to nucleic acid positions 1-60 of SEQ ID NO: 1. In some embodiments, the nucleic acid sequence described herein is connected to a human 5′ UTR. In some embodiments, the nucleic acid sequence described herein is connected to an endogenous 3′ UTR. In some embodiments, the nucleic acid sequence described herein is connected to a human 3′ UTR.

In some embodiments the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire mouse IL33 nucleotide sequence (e.g., NM_133775.3 (SEQ ID NO: 1); NM_001164724.2; or NM_001360725.1).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL33 nucleotide sequence (e.g., exon 1, a portion of exon 2, and a portion of exon 8, of NM_133775.3 (SEQ ID NO: 1); NM_001164724.2; or NM_001360725.1).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human IL33 nucleotide sequence (e.g., NM_033439.3 (SEQ ID NO: 3); NM_001314044.1; NM_001314045.1; NM_001199640.1; NM_001199641.1; NM_1001314046.1; NM_001314047.1; or NM_001314048.1).

In some embodiments, the nucleic acid sequence described herein has at least a portion (e.g., at least 1, 2, 3 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL33 nucleotide sequence (e.g., NM_033439.3 (SEQ ID NO: 3); NM_001314044.1; NM_001314045.1; NM_001199640.1; NM_001199641.1; NM_001314046.1; NM_001314047.1; or NM_001314048.1).

In some embodiments, the amino acid sequence described herein has at least a portion (e.g., at least 1, 2, 3, 4, 5, 6, 7, 8, 9, 10, 11, 12, 13, 14, 15, 20, 30, 40, 60, 70, 80, 90, or 100 amino acid residues, e.g., contiguous or non-contiguous amino acid residues) that is different from a portion of or the entire mouse IL33 amino acid sequence (e.g., NP_598536.2 (SEQ ID NO: 2); NP_001158196.1; or NP_001347654.1).

In some embodiments, the amino acid sequence has at least a portion ., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL33 amino acid sequence (e.g., NP_598536.2 (SEQ ID NO: 2); NP_001158196.1; or NP_001347654.1).

In some embodiments, the amino acid sequence has at least a portion (e.g., at least 1, 2, 4, 5, 6, 7, 8, 9, 10, 11, 12, 13, 14, 15, 20, 30, 40 50, 60, 70, 80, 90, or 100 amino acid residues, e.g., contiguous or non-contiguous amino acid residues) that is different from a portion of or the entire human IL33 amino acid sequence (e.g., NP_254274.1 (SEQ ID NO: 4); NP_001300973.1 (SEQ ID NO: 8); NP_001300974.1 (SEQ ID NO: 8); NP_001186569.1 (SEQ ID NO: 9); NP_001186570.1 (SEQ ID NO: 10); NP_001300975.1 (SEQ ID NO: 11); NP_001300976.1 (SEQ ID NO: 11); or NP_001300977.1 (SEQ ID NO: 12)).

In some embodiments, the amino acid sequence has at least a portion (e.g., at least 1, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL33 amino acid sequence (e.g., NP_254274.1 (SEQ ID NO: 8); NP_001300973.1 (SEQ ID NO: 8); NP_001300974.1 (SEQ ID NO: 8); NP_001186569.1 (SEQ ID NO: 9); NP_001186570.1 (SEQ ID NO: 10); NP_001300975.1 (SEQ ID NO: 11); NP_0013009761 (SEQ ID NO: 11); or NP_001300977.1 (SEQ ID NO: 12)).

The present disclosure also provides a human or humanized IL33 amino acid sequence, wherein the amino acid sequence is selected from the group consisting of:

a) an amino acid sequence shown in SEQ ID NOS: 8-12;

b) an amino acid sequence having a homology of at least 90% with or at least 90% identical amino acid sequence shown in SEQ ID NOS: 8-12;

c) an amino acid sequence encoded by a nucleic acid sequence, wherein the nucleic acid sequence is able to hybridize to a nucleotide sequence encoding the amino acid shown in SEQ ID NOS: 8-12, under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NOS: 8-12;

e) an amino acid sequence that is different from the amino acid sequence shown in SEQ ID NOS: 8-12, by no more than 10, 9, 6, 5, 4, 3, 2 or no more than 1 amino acid; or

f) an amino acid sequence that comprises a substitution, a deletion and/or insertion of one or more amino acids to the amino acid sequence shown in SEQ ID NOS: 8-12.

The present disclosure also relates to a IL33 nucleic acid (e.g. DNA or RNA) sequence, wherein the nucleic acid sequence can be selected from the group consisting of:

a) a nucleic acid sequence as shown in SEQ ID NO: 7, or a nucleic acid sequence encoding a homologous IL33 amino acid sequence of a humanized mouse IL33;

b) a nucleic acid sequence that is shown in SEQ ID NO: 7;

c) a nucleic acid sequence that is able to hybridize to the nucleotide sequence as shown in SEQ ID NO: 7 under a low stringency condition or a strict stringency condition;

d) a nucleic acid sequence that has a homology of at least 90%, 91%, 92%, 93%, 94%, 95%, 96%, 97%, 98%, 99%, or at least 90%, 91%, 92%, 93%, 94%, 95%, 96%, 97%, 98%, or 99% identical to the nucleotide sequence as shown in SEQ ID NO: 7;

e) a nucleic acid sequence that encodes an amino acid sequence, wherein the amino acid sequence has a homology of at least 90% with or at least 90% identical to the amino acid sequence shown in SEQ ID NO: 7;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 7;

g) a nucleic acid sequence that encodes an amino acid sequence, wherein the amino acid sequence is different from the amino acid sequence shown in SEQ ID NO: 7 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 7.

The present disclosure further relates to an IL33 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 7.

The disclosure also provides an amino acid sequence that has a homology of at least 90% with, or at least 90% identical to the sequence shown in SEQ ID NOS: 8-12, and has protein activity. In some embodiments, the homology with the sequence shown in SEQ ID NOS: 8-12, is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NOS: 8-12, is at least about 90%, 91%, 92%, 93%, 94%, 95%, 96%, 97%, 98%, or at least 99%. ln some embodiments, the foregoing percentage identity is at least about 60%, 61%, 62%, 63%, 64% 65% 66, 67% 68% 69% 0%71%72, 73%74% 75% 80% or 85%.,%, , , ,%, , , ,

The disclosure also provides a nucleotide sequence that has a homology of at least 90%, or at least 90% identical to the sequence shown in SEQ ID NO: 7, and encodes a polypeptide that has IL33 protein activity. In some embodiments, the homology with the sequence shown in SEQ ID NO: 7 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ D NO: 7 is at least about 90%, 91%, 92%, 93%, 94%, 95%, 96%, 97%, 98%, or at least 99%. In some embodiments, the foregoing percentage identity least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15©, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or iii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For illustration purposes, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided hat comprise the nucleotide sequences as described herein, as well as cells, tissues, and animals (e.g., mouse) that express human or chimeric (e.g., humanized) IL33 from an endogenous non-human IL33 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous IL33 locus that comprises an exogenous sequence (e.g., a human sequence e.g., a replacement of one or more non-human sequences with one or more human sequences. The animals are generally able to pass the modification to progeny, i.e., through germline transmission.

As used herein, the term “chimeric gene” “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. in some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized IL33 gene or a humanized IL33 nucleic acid. In some embodiments, at least one or more portions of the gene or the nucleic acid is from the human IL33 gene. In some embodiments, the gene or the nucleic acid comprises a sequence that encodes a human or humanized IL33 protein. The encoded IL33 protein is functional or has at least one activity of the human IL33 protein and/or the non-human IL33 protein, e.g., interacting with human or non-human ST2 (or IL1RL1) and/or IL1RAcP; recruiting mast cells and neutrophils into the tumor microenvironment; inducing helper T cells (e.g., polarized Th2 cells), mast cells, eosinophils and/or basophils to produce type 2 cytokines (e.g., IL-5 and IL-13); activating intracellular molecules in the NE-κB and MAP kinase signaling pathways; promoting myeloid-derived suppressor cells; intervening toward CD8+ T, natural killer (NK) cell infiltration, group 2 innate lymphoid cell (ILC2) proliferation, dendritic cell (DC) activation; inhibiting tumor growth and/or metastasis; reversing buildup and preventing new formation of amyloid plaques; and/or upregulating the immune response.

In some embodiments, the chimeric protein or the chimeric polypeptide is a humanized IL33 protein or a humanized IL33 polypeptide, In some embodiments, at least one or more portions of the amino acid sequence of the protein or the polypeptide is from a human IL33 protein. The human IL33 protein or the humanized IL33 protein is functional or has at least one activity of the human IL33 protein or the non-human IL33 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calornyscidae (e.g., mouse-like hamsters), Cricetidae (e.g., hamster, New World rats and mice, voles), Muridae (true mice and rats, gerbils, spiny mice, crested rats), Nesornyidae (climbing mice, rock mice, with-tailed rats, Malagasy rats and mice), Platacanthornyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6M, C57BL/10, C57BL/10ScSn, C57BL/10Cr, and C57BL/O1a. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL33 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL33 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature IL33 coding sequence with human mature IL33 coding sequence.

Genetically modified non-human animals that comprise a modification of an endogenous non-human IL33 locus. In some embodiments, the modification can comprise a human nucleic acid sequence encoding at least a portion of a mature IL33 protein (e.g., at least 10%, 20%, 30%, 40%, 50%, 60%, 70%, 80%, 90%, 95%, 96%, 97%, 98%, 99% or 100% identical to the mature IL33 protein sequence). Although genetically modified cells are also provided that can comprise the modifications described herein (e.g., ES cells, somatic cells), in many embodiments, the genetically modified non-human animals comprise the modification of the endogenous IL33 locus in the germline of the animal.

Genetically modified animals can express a human IL33 and/or a chimeric (e.g., humanized) IL33 from endogenous mouse loci, wherein the endogenous mouse IL33 gene has been replaced with a human IL33 gene and/or a nucleotide sequence that encodes a region of human IL33 sequence or an amino acid sequence that is at least 10%, 20%, 30%, 40%, 50%, 60%, 70%, 80%, 90%, 95%, 96%, 97%, 98%, or 99% identical to the human IL33 sequence. In various embodiments, an endogenous non-human IL33 locus is modified in whole or in part to comprise human nucleic acid sequence encoding at least one protein-coding sequence of a mature IL33 protein.

In some embodiments, the genetically modified mice express the human IL33 and/or chimeric IL33 (e.g., humanized IL33) from endogenous loci that are under control of mouse promoters and/or mouse regulatory elements. The replacement(s) at the endogenous mouse loci provide non-human animals that express human IL33 or chimeric IL33 (e.g., humanized IL33) in appropriate cell types and in a manner that does not result in the potential pathologies observed in some other transgenic mice known in the art. The human IL33 or the chimeric IL33 (e.g., humanized IL33) expressed in animal can maintain one or more functions of the wild-type mouse or human IL33 in the animal. For example, human or non-human IL33 receptors (e.g., ST2) can bind to the expressed IL33, and trigger an inflammatory cascade. Furthermore, in some embodiments, the animal does not express endogenous IL33. As used herein, the term “endogenous IL33” refers to IL33 protein that is expressed from an endogenous IL33 nucleotide sequence of the non-human animal (e.g., mouse) before any genetic modification.

The genome of the animal can comprise a sequence encoding an amino acid sequence that is at least 70%, 80%, 85%, 90%, 95%, 99%, or 100% identical to human IL33 (e.g., NP_254274.1 (SEQ ID NO: 8), NP_001300973.1 (SEQ ID NO: 8); NP_001300974.1 (SEQ ID NO: 8); NP_001186569.1 (SEQ ID NO: 9); NP_001186570.1 (SEQ ID NO: 10); NP_001300975.1 (SEQ ID NO: 11); NP_001300976.1 (SEQ ID NO: 11); or NP_001300977.1 (SEQ ID NO: 12)). In some embodiments, the genome comprises a sequence encoding an amino acid sequence that is at least 70%, 75%, 80%, 85%, 90%, 95%, 99%, or 100% identical to SEQ ID NO: 8-12.

The genome of the genetically modified animal can comprise a replacement at an endogenous IL33 gene locus of a sequence encoding a region of endogenous IL33 with a sequence encoding a corresponding region of human IL33. In some embodiments, the sequence that is replaced is any sequence within the endogenous IL33 gene locus, exon 1, exon 2, exon 3, exon 4, exon 5, exon 6, exon 7, exon 8, 5′-UTR, 3′-UTR, the first intron, the second intron, and the third intron, the fourth intron, the fifth intron, the sixth intron, the seventh intron, etc. In some embodiments, the sequence that is replaced is within the regulatory region of the endogenous IL33 gene. In some embodiments, the sequence that is replaced is exon 2, exon 3, exon 4, exon 5, exon 6, exon 7, exon 8, or a part thereof, of an endogenous mouse IL33 gene locus. In some embodiments, the sequence that is replaced is from a portion of exon 2 to a portion of exon 8 of an endogenous mouse IL33 gene locus. In some embodiments, the sequence that is replaced is from exon 2 to exon 8 of an endogenous mouse IL33 gene locus.

In some embodiments, the genetically modified animal does not express endogenous IL33. In some embodiments, the genetically modified animal expresses a decreased level of endogenous IL33 as compared to a wild-type animal.

Furthermore, the genetically modified animal can be heterozygous with respect to the replacement at the endogenous IL33 locus, or homozygous with respect to the replacement at the endogenous IL33 locus.

In some embodiments, the humanized IL33 locus lacks a human IL33 5′-UTR. In some embodiment, the humanized IL33 locus comprises a rodent (e.g., mouse) 5′-UTR. In some embodiments, the humanization comprises a human 3′-UTR. In some embodiments, the humanization comprises a mouse 3′-UTR. In appropriate cases, it may be reasonable to presume that the mouse and human IL33 genes appear to be similarly regulated based on the similarity of their 5′-flanking sequence. As shown in the present disclosure, humanized IL33 mice that comprise a replacement at an endogenous mouse IL33 locus, which retain mouse regulatory elements but comprise a humanization of IL33 encoding sequence, do not exhibit pathologies. Both genetically modified mice that are heterozygous or homozygous for humanized IL33 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized IL33 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relate^{,,} to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL33 in the genome of the animal.

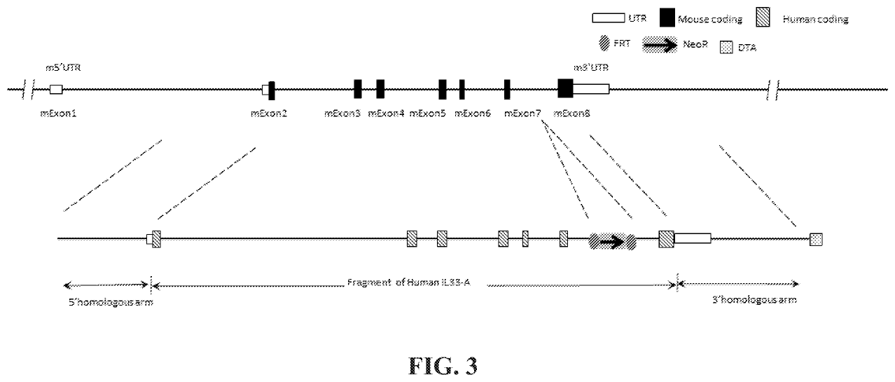

In some embodiments, the non-human mammal comprises the genetic construct as described herein (e.g., gene construct as shown in FIG. 3). In some embodiments, a non-human mammal expressing human or humanized IL33 is provided. In some embodiments, the tissue-specific expression of human or humanized IL33 protein is provided.

In some embodiments, the expression of human or humanized IL33 in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human IL33 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL33 protein.

### Vectors

The present disclosure relates to a targeting vector, comprising: a) a DNA fragment homologous to the 5′ end of a region to be altered (5′ arm), which is selected from the IL33 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL33 gene genomic DNAs in the length of 100 to 10,000 nucleotides,

In some embodiments, a) the DNA fragment homologous to the 5′ end of a conversion region to be altered (5′ arm) is selected from the nucleotide sequences that have at least 90% homology to the NCBI accession number NC_3′ end of the region to be altered (3′ arm) is selected from the nucleotide sequences that have at least 90% homology to the NCBI accession number NC_000085.6.

In some embodiments, a) the DNA fragment homologous to the 5′ end of a region to be altered (5′ arm) is selected from the nucleotides from the position 29945453 to the position 29949670 of the NCBI accession number NC_000085.6; c) the DNA fragment homologous to the 3′ end of the region to be altered (3′ arm) is selected from the nucleotides from the position 29959042 to the position 29963122 of the NCBI accession number NC_000085.6.

In some embodiments, the length of the selected genomic nucleotide sequence in the targeting vector can be more than about 1 kb, about 1.5 kb, about 2 kb, about 2.5 kb, 3 kb, about 3.5 kb, about 4 kb, about 4.5 kb, about 5 kb, about 5.5 kb, or about 6 kb.

In some embodiments, the region to be altered is exon 1, exon 2, exon 3, exon 4, exon 5, exon 6, exon 7, and/or exon8 of IL33 gene (e.g., exon 2, exon 3, exon 4, exon 5, exon 6, exon 7, and/or exon 8 of mouse IL33 gene).

The targeting vector can further include a selected gene marker. In some embodiments, the sequence of the 5′ arm is shown in SEQ ID NO: 5; and the sequence of the 3′ arm is shown in SEQ ID NO: 6.

In some embodiments, the sequence is derived from human (e.g., 6241695-6256168 of NC_000009.12). For example, the target region in the targeting vector is a part or entirety of the nucleotide sequence of a human IL33, preferably exon 2, exon 3, exon 4, exon 5, exon 6, exon 7, and/or exon 8 of the human IL33. In some embodiments, the nucleotide sequence of the humanized. IL33 encodes the entire or the part of human IL33 protein with the NCBI accession number NP_254274.1 (SEQ ID NO: 8); NP_001300973.1 (SEQ ID NO: 8); NP_001300974.1 (SEQ ID NO: 8); NP_001186569.1 (SEQ ID NO: 9); NP_001186570.1 (SEQ ID NO: 10); NP_001300975,1 (SEQ ID NO: 11); NP_001300976.1 (SEQ ID NO: 11); or NP_001300977.1 (SEQ ID NO: 12).

The disclosure also relates to a cell comprising the targeting vectors as described above. In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can made by several techniques that are known in the art, including, e.g., non-homologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL33 gene locus, a sequence encoding a region of an endogenous IL33 with a sequence encoding a corresponding region of human or chimeric IL33. In some embodiments, the replacement occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse IL33 locus. In FIG. 3, the targeting strategy involves a vector comprising the 5′ end homologous arm, human IL33 gene fragment, 3′ homologous arm. The process can involve replacing endogenous IL33 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL33 sequence with human IL33 sequence.

Thus, in some embodiments, the methods fir making a genetically modified, humanized animal, can include the step of replacing at an endogenous IL33 locus site), a nucleic acid encoding a sequence encoding a region of endogenous IL33 with a sequence encoding a corresponding region of human IL33. The sequence can include a region (e.g., a part or the entire region) of exon 1, exon 2, exon 3, exon 4, exon 5, exon 6, exon 7, and/or exon 8 of a human IL33 gene. In some embodiments, the sequence includes a region of exon 2, exon 3, exon 4, exon 5, exon 6, exon 7, and exon 8 of a human IL33 gene (e.g., a sequence encoding amino acids 1-270 of SEQ ID NO: 8). In some embodiments, the endogenous IL33 locus is exon 2, exon exon 4, exon exon 6, exon 7, and/or exon 8 of mouse IL33 gene (e.g., a sequence encoding amino acids 1-266 of SEQ ID NO: 2).

In some embodiments, the methods of modifying a IL33 locus of a mouse to express a chimeric human/Mouse IL33 peptide can include the steps of replacing at the endogenous mouse IL33 locus a nucleotide sequence encoding a mouse IL33 with a nucleotide sequence encoding a human IL33, thereby generating a sequence encoding a chimeric human/mouse IL33.

In some embodiments, the nucleotide sequences as described herein do not overlap with each other (e.g., the 5′ homologous arm, the IL33-A fragment, and/or the 3′ homologous arm do not overlap). In some embodiments, the amino acid sequences as described herein do not overlap with each other.

The present disclosure further provides a method for establishing a IL33 gene humanized. animal model, involving the following steps:

(a) providing the cell (e.g. an embryonic stem cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human ⁻mammal in the foregoing method is a mouse (e.g., a C57BL/6 mouse).

In some embodiments, the non-human mammal in step (c) is a female with pseudo pregnancy (or false pregnancy).

In some embodiments, the embryonic stem cells for the methods described above are C57BL/6 embryonic stein cells. Other embryonic stem cells that can also be used in the methods as described herein include, but are not limited to, FVB/N embryonic stem cells, BALB/c embryonic stem cells, DBA/1 embryonic stem cells and DBA/2 embryonic stein cells.

Embryonic stem cells can come from any non-human animal, e.g., any non-human animal as described herein. In some embodiments, the embryonic stem cells are derived from rodents. The genetic construct can be introduced into an embryonic stem cell by microinjection of DNA. For example, by way of culturing an embryonic stem cell after microinjection, a cultured embryonic stem cell can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL33 protein, e.g., in a physiologically appropriate manner, provide a variety of uses that include, but are not limited to, developing therapeutics fir human diseases and disorders, and assessing the toxicity and/or the efficacy of these human therapeutics in the animal models.

In various aspects, genetically modified animals are provided that express human or humanized IL33, which are useful for testing agents that can decrease or block the interaction between IL33 and IL33 receptors (e.g., IL1RL1) or the interaction between IL33 and anti-human IL33 antibodies, testing whether an agent can increase or decrease the immune response, and/or determining whether an agent is an IL33 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In one aspect, the disclosure also provides methods of determining effectiveness of an IL33 antagonist (e.g., an anti-IL33 antibody) for reducing inflammation. The methods involve administering the IL33 antagonist to the animal described herein, wherein the animal has an inflammation; and determining effects of the IL33 antagonist for reducing the inflammation.

In one aspect, the disclosure also provides methods of determining effectiveness of an IL33 antagonist (e.g., an anti-IL33 antibody) for treating an immune disorder (e.g., an autoimmune disorder or allergic disorder). The methods involve administering the IL33 antagonist to the animal described herein, wherein the animal has an immune disorder; and determining effects of the IL33 antagonist for treating the immune disorder.

In some embodiments, the effects of reducing the inflammation, or treating the immune disorder are evaluated by serum IgE levels; pathological lung histology features; number of leukocytes (CD45+ cells), eosinophils (Eos) or neutrophils in bronchoalveolar lavage fluid (BALF); or percentages of eosinophils or neutrophils cells in CD45+ cells in bronchoalveolar lavage fluid (BALF).

In some embodiments, the genetically modified animals can be used for determining effectiveness of an anti-IL33 antibody for treating cancer. The methods involve administering the anti-IL33 antibody (e.g., anti-human IL33 antibody) to the animal as described herein, wherein the animal has a tumor; and determining the inhibitory effects of the anti-IL33 antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in minor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the IL33 antibody is a monoclonal antibody. In some embodiments, the IL33 antibody is REGN-3500 (SAR440340). Details of REGN-3500 can be found, e.g., in WO2018102597A1, which is incorporated herein by reference in its entirety. In some embodiments, the IL33 antibody is Antibody 43. Details of Antibody 43 can be found, e.g., in WO2018081075A1, which is incorporated herein by reference in its entirety.

In some embodiments, the tumor comprises one or more cancer cells (e.g., human or mouse cancer cells) that are injected into the animal. In some embodiments, the anti-IL33 antibody or anti-ST2 antibody prevents ST2 from binding to IL33. In some embodiments, the anti-IL33 antibody or anti-ST2 antibody does not prevent ST2 from binding to IL33.

In some embodiments, the genetically modified animals can be used for determining whether an anti-IL33 antibody is a IL33 agonist or antagonist. In some embodiments, the methods as described herein are also designed to determine the effects of the agent (e.g., anti-IL33 antibodies) on IL33, e.g., interacting with ST2, or inducing type 2. cytokine release. In some embodiments, the genetically modified animals can be used for determining the effective dosage of a therapeutic agent for treating a disease in the subject, e.g., an immune disorder, an allergy, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV}(%)=(I−TVt/TVc)×100, where TVt and TVc are the mean tumor volume (or weight) of treated and control groups.

In some embodiments, the anti-IL33 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the anti-IL33 antibody designed for treating breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, or bone cancer. In some embodiments, the anti-IL33 antibody is designed for treating solid tumor. In some embodiments, the anti-IL33 antibody is designed for treating metastatic solid tumors. In some embodiments, the anti-IL33 antibody is designed for reducing tumor growth, metastasis, and/or angiogenesis.

In some embodiments, the antibody is designed for treating various autoimmune diseases or allergy (e.g., allergic rhinitis, sinusitis, asthma, rheumatoid arthritis, atopic dermatitis, chronic obstructive pulmonary disease (COPD), chronic bronchitis, emphysema, or eczema). Thus, the methods as described herein can be used to determine the effectiveness of an antibody in inhibiting immune response.

In some embodiments, the antibody is designed for reducing inflammation (e.g., inflammatory bowel disease, chronic inflammation, asthmatic inflammation, or wound healing). Thus, the methods as described herein can be used to determine the effectiveness of an antibody for reducing inflammation. In some embodiments, the antibody is designed for treating other diseases (e.g., endometriosis).

The present disclosure also provides methods of determining toxicity of an antibody (e.g., anti-IL33 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL33 gene function, human IL33 antibodies, drugs for human IL33 targeting sites, the drugs or efficacies for human IL33 targeting sites, the drugs for immune-related diseases and antitumor drugs.

In some embodiments, the disclosure provides a method to verify in vivo efficacy of TCR-T , CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL33 gene humanized non-human animal prepared by the methods described herein, the IL33 gene humanized non-human animal described herein, the double- or multi-humanized non-human animal generated by the methods described herein (or progeny thereof), a non-human animal expressing the human or humanized IL33 protein, or the tumor-bearing or inflammatory animal models described herein. In some embodiments, the TCR-T, CAR-T, and/or other immunotherapies can treat the IL33-associated diseases described herein. In some embodiments, the TCA-T, CAR-T, and/or other immunotherapies provides an evaluation method for treating the IL33-associated diseases described herein.

### Genetically Modified Animal Model With Two or More Human or Chimeric Genes

The present disclosure further relates to methods for generating genetically modified animal model with two or more human or chimeric genes. The animal can comprise a human or chimeric IL33 gene and a sequence encoding an additional human or chimeric protein.

In some embodiments, the additional human or chimeric protein can be programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand I (PD-L1), CD3, CD27, CD28, CD47, CD137, CD154, T-Cell Immunoreceptor With Ig And ITIM Domains (TIGIT), T-cell immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

The methods of generating genetically modified animal model with two or more human or chimeric genes (e.g., humanized genes) can include the following steps:

(a) using the methods of introducing human IL33 gene or chimeric IL33 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, IL15 receptor, BTLA PD-L1, CD3, CD27, CD28, CD47, CD137, CD154, TIGIT, TIM-3, GITR, SIRPα or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the IL33 humanization is directly performed on a genetically modified animal having a human or chimeric PD-1, LAG-3, IL15 receptor, BRA, PD-L1, CD3, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL33 antibody and an additional therapeutic agent for the treatment of cancer or an immune disorder. The methods include administering the anti-IL33 antibody and the additional therapeutic agent to the animal, wherein the animal has a tumor; and determining the inhibitory effects of the combined treatment to the tumor. In some embodiments, the additional therapeutic agent is an antibody that specifically binds to PD-1, CTLA-4, LAG-3, IL15 receptor, BTLA, PD-L1, CD3, CD27, CD28, CD47, CD137, CD154, TIGIT, TIM-3, GITR, SIRPα or OX40. In some embodiments, the additional therapeutic agent is an anti-CTLA4 antibody (e.g., ipilimuniab), an anti-PD-1 antibody (e.g., nivolumab), or an anti-PD-L1 antibody.

In some embodiments, the animal further comprises a sequence encoding a human or humanized a sequence encoding a human or humanized PD-L1, or a sequence encoding a human or humanized CTLA-4. in some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, or bone cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

C57BL/6 mice and Flp mice e purchased from the China Food and Drugs Research Institute National Rodent Experimental Animal Center.

Cas9 mRNA was obtained from SIGMA (Catalog Number: CAS9MRNA-1EA).

UCA kit was obtained from Beijing Biocytogen Co., Ltd. (Catalog Number: BCG-DX-001).

MEGAshortscript™ Kit (Ambion in vitro transcription kit) was purchased from Thermo Fisher Scientific Inc. (Catalog Number: AM1354).

Lipopolysaccharides from Escherichia coli O111:B4 (LPS) was purchased from Merck (Catalog Number: L2630).

LEGEND MAX™ Mouse IL-33 ELISA Kit with Pre-coated Plates (mouse IL33 kit) were purchased from BioLegend, Inc. (Catalog Number: 436407).

LEGEND MAX™ Human IL-33 ELISA Kit with Pre-coated Plates (human kit) were purchased from BioLegend, Inc. (Catalog Number: 435907).

SacI, PstI, SpeI, HindIII, XbaI, and ScaI restriction enzymes were purchased from NEB with Catalog numbers: R3156M, R3140M, R3133M, R3104M, R3122M, R3122M, respectively.

### Example 1: Mice with Humanized IL33 Gene

The mouse IL33 gene (NCBI Gene ID: 77125, Primary source: MGI: 1924375, UniProt ID: Q8BVZ5) is located at 29925114 to 29960715 of chromosome 19 (NC_000085.61, and the human IL33 gene (NCBI Gene ID: 90865, Primary source: HGNC: 16028, UniProt ID: 095760) is located at 6214591 to 6257983 of chromosome 9 (NC_000009.12). FIG. 1A shows the mouse transcript NM_133775.3 (SEQ ID NO: 1) and the corresponding protein sequence NP_598536.2 (SEQ ID NO: 2); and FIG. 1B shows the human transcript NM_033439.3 (SEQ ID NO: 3) and the corresponding protein sequence NP_254274.1 (SEQ ID NO: 8).

A gene sequence encoding the human IL33 protein can be introduced into the endogenous mouse IL33 locus, such that the mouse can express a human or humanized IL33 protein. Mouse cells can be modified by various gene-editing techniques, for example, replacement of specific mouse IL33 gene sequences with human IL33 gene sequences at the endogenous mouse IL33 locus. For example, a sequence about 9371 by spanning from exon 2 to exon 8 of mouse IL33 gene was replaced with a corresponding human DNA sequence to obtain a humanized IL33 locus, thereby humanizing mouse IL33 gene (shown in FIG. 2).

As shown in the schematic diagram of the targeting strategy in FIG. 3, the targeting vector contained homologous arm sequences upstream and downstream of mouse IL33 gene locus (about 4218 by upstream of a portion of exon 2 and about 4081 by downstream of a portion of exon 8 of endogenous IL33 gene), and an “IL33-A fragment” comprising a human IL33 gene sequence. The upstream homologous arm sequence (5′ homologous arm, SEQ ID NO: 5) is identical to nucleotide sequence of 29945453-29949670 of NCBI accession number NC_000085.6, and the downstream homologous arm sequence (3′ homologous arm, SEQ ID NO: 6) is identical to nucleotide sequence of 29959042-29963122 of NCBI accession number NC_000085.6. The IL33-A fragment comprises a genomic DNA sequence from a portion of exon 2 to a portion of exon 8 of human IL33 gene, which is identical to nucleotide sequence of 6241695-6256168 with NCBI accession number NC_000009.12.

The modified humanized mouse IL33 mRNA sequence is shown in SEQ ID NO: 7, and the expressed protein has the same sequence as human IL33 protein shown in SEQ ID NO: 8. Given that human IL33 and mouse IL33 have multiple isoforms or transcripts, the methods described herein can be applied to other isoforms or transcripts. In other words, the modified humanized mouse can express all human or humanized IL33 protein sequences. The transcripts of IL33 gene (mouse and human), and the expressed humanized IL33 protein sequences are summarized in Table 3 below.

TABLE 3
Sequence of IL33 protein expressed by
humanized mice from different transcripts

| | | Sequence of IL33 protein expressed by |
|---|---|---|
| Human transcripts | Mouse transcripts | humanized mice |
| NM_033439.3→ | NM_133775.3→ | MKPKMKYSTNKISTAKWKNTASKALCFKLGKSQQKAKEVC |
| NP_254274.1 | NP_598536.2 | PMYFMKLRSGLMIKKEACYFRRETTKRPSLKTGRKHKRHLV |
| NM_001314044.1 → | NM_001164724.2→ | LAACQQQSTVECFAFGISGVQKYTRALHDSSITGISPITEYLA |
| NP_001300973.1 | NP_001158196.1 | SLSTYNDQSITFALEDESYEIYVEDLKKDEKKDKVLLSYYESQ |
| NM_001314045.1 → | NM_001360725.1→ | HPSNESGDGVDGKMLMVTLSPTKDFWLHANNKEHSVELH |
| NP_001300974.1 | NP_001347654.1 | KCEKPLPDQAFFVLHNMHSNCVSFECKTDPGVFIGVKDNH |
| | | LALIKVDSSENLCTENILFKLSET (SEQ ID NO: 8) |
| NM_001199640.1 → | NM_133775.3→ | MKPKMKYSTNKISTAKWKNTASKALCFKLGKSQQKAKEVC |
| NP_001186569.1 | NP_598536.2 | PMYFMKLRSGLMIKKEACYFRRETTKRPSLKTGRKHKRHLV |
| | NM_001164724.2→ | LAACQQQSTVECFAFGISGVQKYTRALHDSSITDKVLLSYYE |
| | NP_001158196.1 | SQHPSNESGDGVDGKMLMVTLSPTKDFWLHANNKEHSV |
| | NM_001360725.1→ | ELHKCEKPLPDQAFFVLHNMHSNCVSFECKTDPGVFIGVKD |
| | NP_001347654.1 | NHLALIKVDSSENLCTENILFKLSET (SEQ ID NO: 9) |
| NM_001199641.1 → | NM_133775.3→ | MKPKMKYSTNKISTAKWKNTASKALCFKLGNKVLLSYYESQ |
| NP_001186570.1 | NP_598536.2 | HPSNESGDGVDGKMLMVTLSPTKDFWLHANNKEHSVELH |
| | NM_001164724.2→ | KCEKPLPDQAFFVLHNMHSNCVSFECKTDPGVFIGVKDNH |
| | NP_001158196.1 | LALIKVDSSENLCTENILFKLSET (SEQ ID NO: 10) |
| | NM_001360725.1→ | |
| | NP_001347654.1 | |
| NM_001314046.1 → | NM_133775.3→ | MKPKMKYSTNKISTAKWKNTASKALCFKLGKSQQKAKEVC |
| NP_001300975.1 | NP_598536.2 | PMYFMKLRSGLMIKKEACYFRRETTKRPSLKTGRKHKRHLV |
| NM_001314047.1 → | NM_001164724.2→ | LAACQQQSTVECFAFGISGVQKYTRALHDSSITEYLASLSTY |
| NP_001300976.1 | NP_001158196.1 | NDQSITFALEDESYEIYVEDLKKDEKKDKVLLSYYESQHPSNE |
| | NM_001360725.1→ | SGDGVDGKMLMVTLSPTKDFWLHANNKEHSVELHKCEKP |
| | NP_001347654.1 | LPDQAFFVLHNMHSNCVSFECKTDPGVFIGVKDNHLALIKV |
| | | DSSENLCTENILFKLSET (SEQ ID NO: 11) |
| NM_001314048.1 → | NM_133775.3→ | MKPKMKYSTNKISTAKWKNTASKALCFKLGKSQQKAKEVC |
| NP_001300977.1 | NP_398536.2 | PMYFMKLRSGLMIKKEACYFRRETTKRPSLKTGISPITEYLAS |
| | NM_001164724.2→ | LSTYNDQSITFALEDESYEIYVEDLKKDEKKDKVLLSYYESQH |
| | NP_001158196.1 | PSNESGDGVDGKMLMVTLSPTKDFWLHANNKEHSVELHK |
| | NM_001360725.1→ | CEKPLPDQAFFVLHNMHSNCVSFECKTDPGVFIGVDNHL |
| | NP_001347654.1 | ALIKVDSSENLCTENILFKLSET (SEQ ID NO: 12) |

The targeting vector also included an antibiotic resistance gene for positive clone screening (neomycin phosphotransferase gene, or Neo), and two Frt recombination sites Hankin the antibiotic resistance gene, that formed a Neo cassette. The Neo cassette is located between exon 7 and exon 8 of human IL33 gene. The connection between the 5′ end of the Neo cassette and the human IL33 sequence was designed as:

(SEQ ID NO: 13)
5′-CATTCTGAGCCTGCTTAAGGGAGAGTCAGTCGACGGTATCGATAAGCT
TGATATCGAATTCCGAAGTTCCT-3′,

wherein he last “A” of the sequence “GAGTCA” is the last nucleotide of the human sequence, and the first “G” of the sequence “GTCGA” is the first nucleotide of the Neo cassette. The connection between the 3′ end of the Neo cassette with the human IL33 sequence was designed as

(SEQ ID NO: 14)
5′-TCCTATTCTCTAGAAAGTATAGGAACTTCATCAGTCAGGTACATAATG
GTGGATCCTAAGCGTTTCCATG-3′,

wherein the last “C” of the sequence “GGATCC” is the last nucleotide of the Neo cassette, and the “T” of the sequence “TAAGC” is the first nucleotide of the human sequence. In addition, a coding gene with a negative selectable marker (a gene encoding diphtheria toxin A subunit (DTA)) was also inserted downstream of the 3′ homologous arm of the targeting vector.

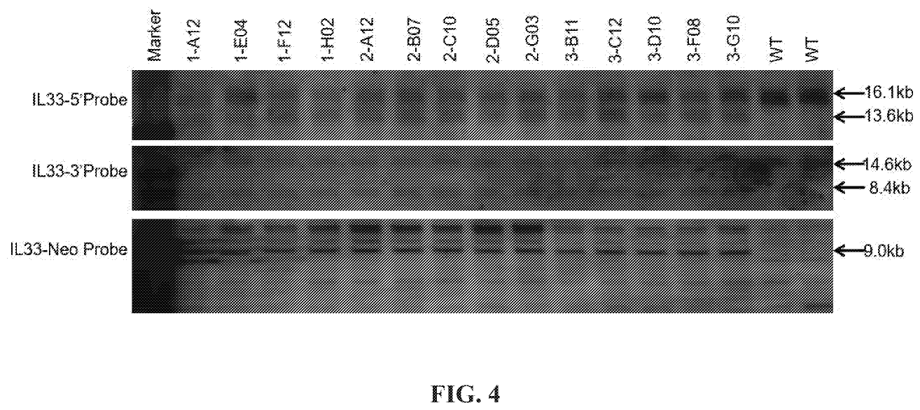

The targeting vector was constructed using standard methods, e.g., by restriction enzyme digestion and ligation. The constructed targeting vector sequence was preliminarily verified by restriction enzyme digestion, followed by verification by sequencing. The correct targeting vector was electroporated and transfected into embryonic stern cells of C57BL/6 mice. The positive selectable marker gene was used to screen the cells, and the integration of exogenous genes was confirmed by PCR and Southern Blot. Specifically, positive clones identified by PCR were further confirmed by Southern Blot (digested with HindIII, XbaI, or ScaI, respectively, and hybridized with 3 probes) to screen out correct positive clone cells. As shown in FIG. 4, the results indicated that among the 14 positive clones confirmed by PCR, all except 1-E04 were positive heterozygous clones and no random insertions were detected.

The following primers were used in PCR:

IL33-F1:
(SEQ ID NO: 15)
5′-GCTCGACTAGAGCTTGCGGA-3′;
IL33-R1:
(SEQ ID NO: 16)
5′-AGAGGCTCTTACAGGGAAGGGGATA-3′;
IL33-F2:
(SEQ ID NO: 17)
5′-AGGAGAAGCCTAGAAAGAGCCCAGT-3′;
IL33-R2:
(SEQ ID NO: 18)
5′-TGCTTGCTGTGTTCTTCCACTTTGC-3′.

The following probes were used Southern Blot assays:

IL33-5′ probe:
5-F:
(SEQ ID NO: 19)
5′-TATAGCTGGTCACGTGGTAGCCTCA-3′,
5-R:
(SEQ ID NO: 20)
5′-ACTGGGCTCTTTCTAGGCTTCTCCT-3′;
IL33-3′ probe:
3-F:
(SEQ ID NO: 21)
5′-AGGCTAGCACTCACCCTTACTCTCC-3′,
3-R:
(SEQ ID NO: 22)
5′-TAGATCGAGAGGTGCACAGTCAAGC-3′;
IL33-Neo probe:
Neo-F:
(SEQ ID NO: 23)
5′-GGATCGGCCATTGAACAAGATGG-3′,
Neo-R:
(SEQ ID NO: 24)
5′-CAGAAGAACTCGTCAAGAAGGCG-3′.

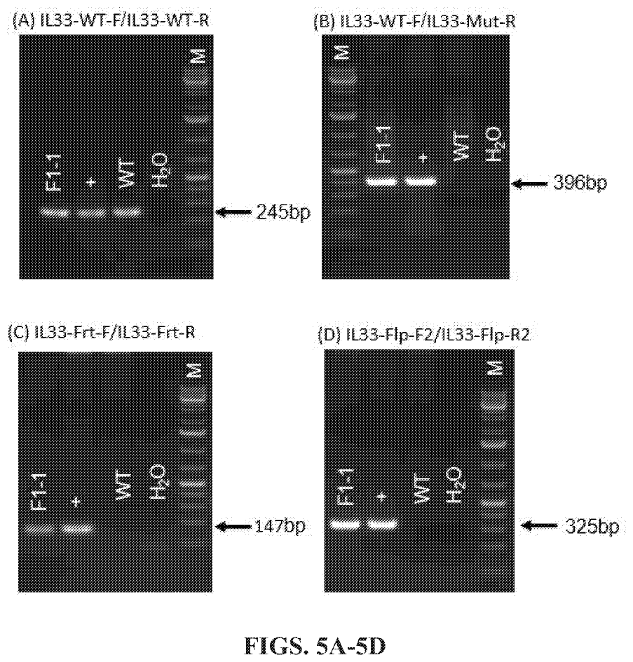

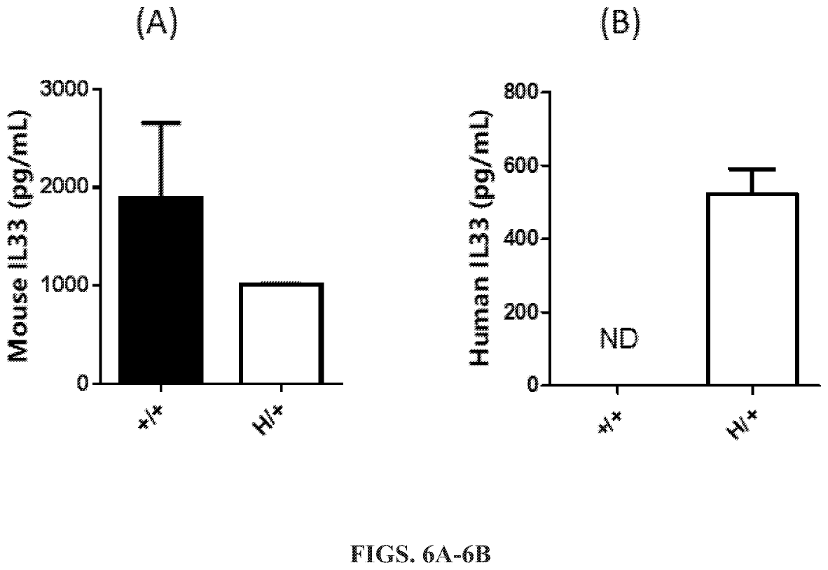

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then mating the F1 generation heterozygous mice with each other. The positive mice were also mated with the Flp mice to remove the positive selectable marker gene, and then the humanized IL33 homozygous mice expressing human IL3 protein were obtained by mating with each other. The genotype of the progeny mice can be identified by PCR. The identification results of exemplary F I generation mice (Neo cassette-removed) are shown in FIGS. 5A-5D, and a mouse labelled F1-1 was identified as a positive heterozygous clone. The following primers were used in the PCR identification:

IL33-WT-F:
(SEQ ID NO: 25)
5′-AGGTTGCTTCTGATGACTTCAGGTCC-3′,
IL33-WT-R:
(SEQ ID NO: 26)
5′-AGGTCGCCCGTCTTCATGTTGAAAT-3′;
IL33-WT-F:
(SEQ ID NO: 25)
5′-AGGTTGCTTCTGATGACTTCAGGTCC-3′,
IL33-Mut-R:
(SEQ ID NO: 27)
5′-TTATCTCAGCTATTCCTGCCTGGTG-3′;
IL33-Frt-F:
(SEQ ID NO: 28)
5′-CCATTCTGAGCCTGCTTAAGGGAGA-3′,
IL33-Frt-R:
(SEQ ID NO: 29)
5′-ATCTTGGCACATGGAAACGCTTAGG-3′;
IL33-Flp-F2:
(SEQ ID NO: 30)
5′-GACAAGCGTTAGTAGGCACATATAC-3′,
IL33-Flp-R2:
(SEQ ID NO: 4)
5′-GCTCCAATTTCCCACAACATTAGT-3′.

The results indicated that this method can be used to construct genetically engineered IL33 mice without random insertions. The expression of humanized IL33 protein in positive clone mice can be confirmed by routine detection methods, such as ELISA. Three 6-7 week old wild-type mice and three IL33 gene humanized heterozygous mice were selected, and 20 ₁.1,g Lipopolysaccharide (LPS) was injected to each mouse intraperitoneally. After 2 hours, mouse lung tissue was collected and grinded to obtain an extract fluid, which was then diluted 30 folds to detect the mouse IL33 and diluted 5 folds to detect human IL33, respectively. As shown in FIG. 6A, expression of mouse IL33 was detected in wild-type C57BL/6 mice and IL33 gene humanized heterozygous mice. However, FIG. 6B shows that human IL33 protein expression was not detected in wild-type C57BL/6 mice, but was detected in IL33 gene humanized heterozygous mice.

### Example 2: Generation and Use of Double- or Multi-Gene Humanized Mice

Double- or multi-gene humanized mice can be obtained by mating the IL33 single-gene humanized mice prepared by the above method with other single-, double-, or multi-gene humanized mice, and positive offspring can be screened. Alternatively, embryonic stem cells or fertilized eggs can be isolated from single-, double-, or multi-gene humanized mice, and processed using methods including homologous recombination, in vitro fertilization (IVF), and/or natural mating, etc., in order to obtain transgenic mice expressing a combination of humanized proteins. Multiple human disease models can be induced/prepared by using the mice described herein. The mice can also be used to test the efficacy of human specific antibodies in vivo. For example, IL33 gene humanized mice can be used to evaluate the pharmacodynamics, pharmacokinetics, and in vivo therapeutic efficacy of IL33 signaling pathway modulators in various disease models known in the art.

### Example 3: Ovalbumin OVA) Combined With Aluminum Hydroxide-Induced Asthma Model

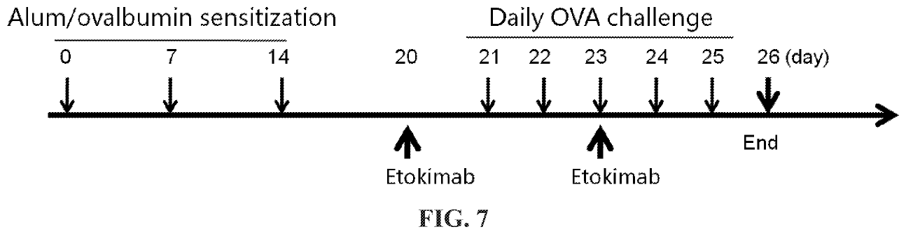

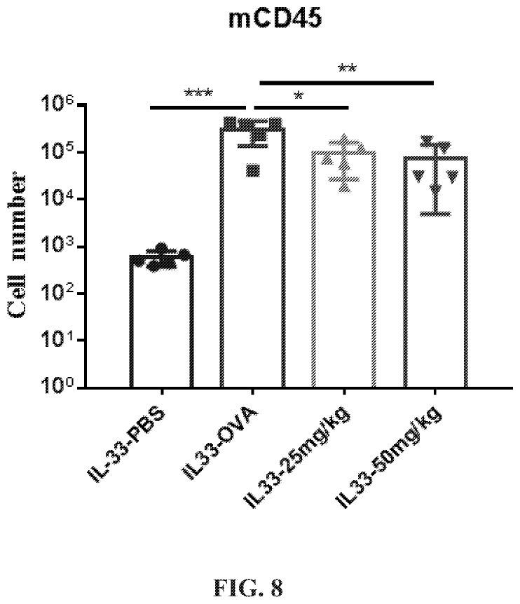

Humanized IL33 gene homozygous mice (or B-hIL33 mice; 5-8 weeks) were selected and randomly divided into four groups (See Table 4). The G3 and G4 group mice were administered with an anti-IL33 antibody Etokimab at 25 mg/kg and 50 mg/kg, respectively. Specifically, the mice in group G2-G4 were exposed 3 times to ovalbumin (OVA) in combination with aluminum hydroxide by intraperitoneal injection. Etokimab was injected intraperitoneally at day 20 and day 23 after the first OVA injection. After 3 weeks of the first OVA injection, 2% OVA aerosols was generated by a nebulizer and was administered continuously for 5 days to make an inducible asthma model (modeling protocol is shown in FIG. 7). In control group G1, phosphate-buffered saline (PBS) was used instead of OVA. All samples were subjected for analysis on day 26.

TABLE 4

| | Mice and | Alum/ovalbumin | Challenge/ | |
|---|---|---|---|---|
| Group | number (n) | sensitization | nebulization | Treatment |
| G1 | B-hIL33 | PBS | PBS | NA |
| | mice (n = 5) | | | |
| G2 | B-hIL33 | Al(OH)₃ + OVA | 2% OVA | NA |
| | mice (n = 5) | | | |
| G3 | B-hIL33 | Al(OH)₃ + OVA | 2% OVA | Etokimab |
| | mice (n = 5) | | | (25 mg/kg) |
| G4 | B-hIL33 | Al(OH)₃ + OVA | 2% OVA | Etokimab |
| | mice (n = 5) | | | (50 mg/kg) |

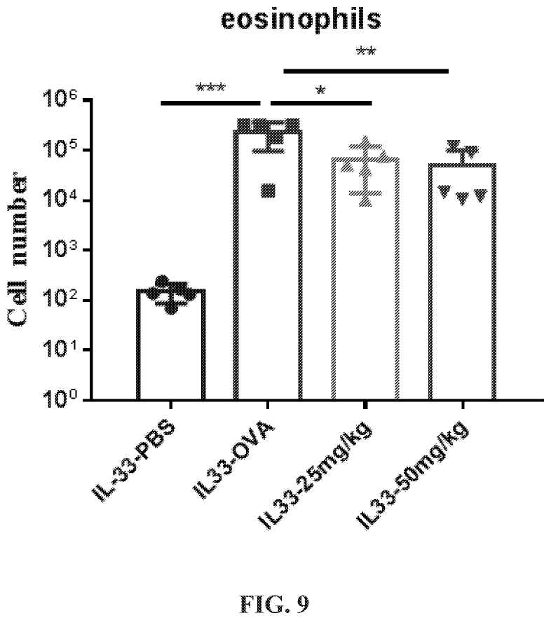

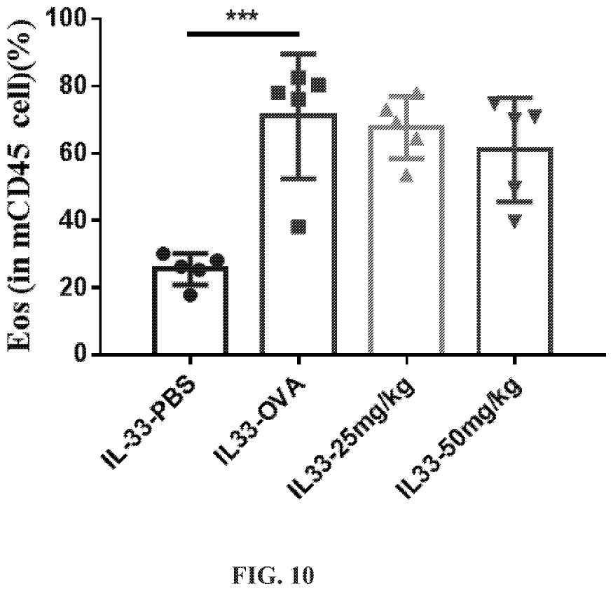

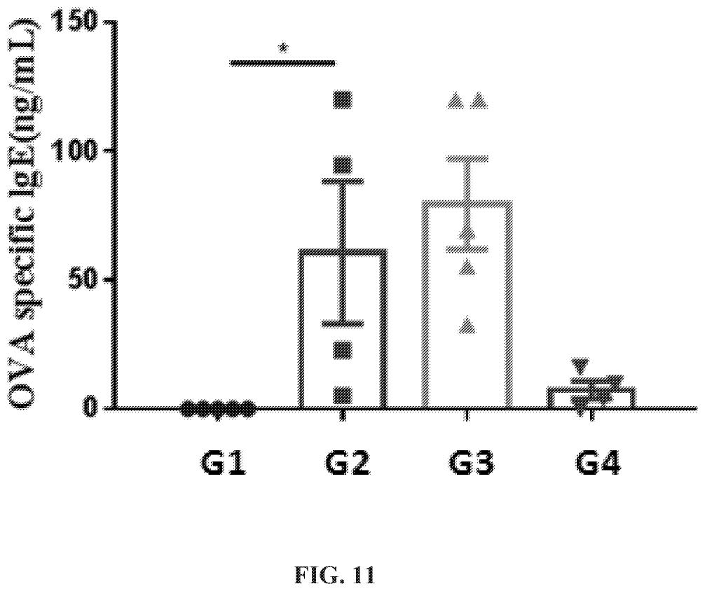

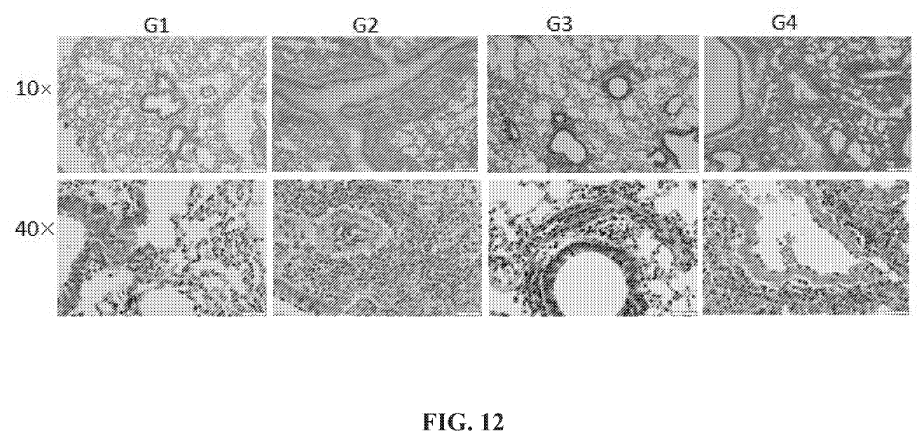

Compared to mice in the G1 control group, the G2 group mice exhibited typical symptoms such as elevated serum IgE levels and pathological lung histology features (FIG. 11 and FIG. 12). infiltrating cell analysis in bronchoalveolar lavage fluid (BALF) suggested a significant decrease of total leukocyte (CD45+ cells) and eosinophils (Eos) cell number in Etokimab-treated mice as compared to the G2 group mice (FIG. 8 and FIG. 9). As shown in FIG. 10, the proportion of Eos cells in CD45+ cells also showed a significant decrease in anti-IL33 antibody-treated mice as compared to the G2 group mice. In addition, FIG. 11 shows that a relatively high serum IgE level was detected in the G2 group mice, and the IgE level decreased in the anti-IL33 antibody-treated group G4 mice. No significant IgE level difference was observed between G2 and G3.

As shown in FIG. 12, H&E staining showed that the airway of the control (G1) (PBS) mice had no inflammation, whereas peribronchial and perivascular inflammation was significantly increased in the OVA-induced group (G2) mice, with increased mucus secretion levels as compared to the control mice (G1). In both of the Etokimab-treatment groups (G3, G4), inflammatory infiltration and mucus secretion were observed (compared to the G2 group) at reduced levels. The results indicate that the humanized IL33 mice prepared by the above method provide a disease model and can be used in preclinical studies to screen and evaluate in vivo efficacy of anti-human IL33 antibodies.

### OTHER EMBODIMENTS

It is to be understood that while the invention has been described in conⱼunction with he detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric IL33.

2. The animal of claim wherein the sequence encoding the human or chimeric IL33 is operably linked to an endogenous regulatory element at the endogenous IL33 gene locus in the at least one chromosome.

3. The animal of claim 1, wherein the sequence encoding a human or chimeric IL33 is operably linked to an endogenous 5′ untranslated region (5′UTR).

4. The animal of any one of claims 1-3, wherein the sequence encoding a human or chimeric IL33 comprises a sequence encoding an amino acid sequence that is at least 70%, 75%, 80%, 85%, 90%, 95%, 99%, or 100% identical to human IL33 (SEQ ID NO: 8, SEQ ID NO: 9, SEQ ID NO: 10, SEQ ID NO: 11, or SEQ ID NO: 12).

5. The animal of claim 4, wherein the sequence encoding a human or chimeric IL33 comprises a sequence encoding an amino acid sequence that is at least 70%, 75%, 80%, 85%, 90%, 95%, 99%, or 100% identical to SEQ ID NO: 8.

6. The animal of claim 4, wherein the sequence encoding a human or chimeric IL33 comprises a sequence encoding an amino acid sequence that is at least 70%, 75%, 80%, 85%, 90%, 95%, 99%, or 100% identical to SEQ ID NO: 9.

7. The animal of claim 4, herein: the sequence encoding a human or chimeric IL33 comprises a sequence encoding an amino acid sequence that is at least 70%, 75%, 80%, 85%, 90%, 95%, 99%, or 100% identical to SEQ ID NO: 10.

8. The animal of claim 4, wherein the sequence encoding a human or chimeric IL33 comprises a sequence encoding an amino acid sequence that is at least 70%, 75%, 80%, 85%, 90%, 95%, 99%, or 100% identical to SEQ ID NO: 11.

9. The animal of claim 4, wherein the sequence encoding a human or chimeric IL33 comprises a sequence encoding an amino acid sequence that is at least 70%, 75%, 80%, 85%, 90%, 95%, 99%, or 100% identical to SEQ ID NO: 12.

10. The animal of any one of claims 1-9, wherein the animal is a mammal, e.g., a monkey, a rodent, or a mouse.

11. The animal of claim 10, wherein the mammal is a mouse.

12. The animal of any one of claims 1-11, wherein the animal does not express endogenous IL33.

13. The animal of any one of claims 1-12, wherein the animal has one or more cells expressing human or chimeric IL33.

14. The animal of claim 13, wherein the expressed human or chimeric IL33 can bind to human interleukin 1 receptor-like 1 (IL1RL1).

15. The animal of claim 13, wherein the expressed human or chimeric IL33 can bind to endogenous IL1RL1.

16. A genetically-modified, non-human animal, wherein the genome of the animal comprises a replacement of a sequence encoding a region of endogenous IL33 with a sequence encoding a corresponding region of human IL33 at an endogenous IL33 gene locus.

17. The animal of claim 16, wherein the sequence encoding the corresponding region of human IL33 is operably linked to an endogenous regulatory element at the endogenous IL33 locus.

18. The animal of claim 16 or 17, wherein the animal does not express endogenous IL33, and animal has one or more cells expressing human or chimeric IL33.

19. The animal of any one of claims 16-18, wherein the animal is a mouse, and the sequence encoding the corresponding region of human IL33 comprises exon 2, exon 3, exon 4, exon 5, exon 6, exon 7 and/or exon 8, or a part thereof, of human IL33 gene.

20. The animal of any one of claims 16-19, wherein the animal is heterozygous with respect to the replacement at the endogenous IL33 gene locus.

21. The animal of any one of claims 16-19, wherein the animal is homozygous with respect to the replacement at the endogenous IL33 gene locus.

22. A method for making a genetically-modified, non-human animal, comprising:
replacing in at least one cell of the animal, at an endogenous IL33 gene locus, a sequence encoding a region of an endogenous IL33 with a sequence encoding a corresponding region of human IL33.

23. The method of claim 22, wherein the sequence encoding the corresponding region of human IL33 comprises exon 2, exon 3, exon 4, exon 5, exon 6, exon 7, and/or exon 8, or a part thereof, of a human IL33 gene.

24. The method of claim 22, wherein the sequence encoding the corresponding region of human IL33 encodes an amino acid sequence that is at least 70%, 75%, 80%, 85%, 90%, 95%, 99%, or 100% identical to SEQ ID NO: 8, SEQ ID NO: 9, SEQ ID NO: 10, SEQ ID NO: 11, or SEQ ID NO: 12.

25. The method of any one of claims 22-24, wherein the animal is a mouse, and the endogenous IL33 locus comprises exon 2, exon 3, exon 4, exon 5, exon 6, exon 7, and/or exon 8, or a part thereof, of the mouse IL33 gene.

26. A non-human animal comprising at least one cell comprising a nucleotide sequence encoding an exogenous IL33 polypeptide, wherein the exogenous IL33 polypeptide comprises at least 50 contiguous amino acid residues that are identical to the corresponding contiguous amino acid sequence of a human IL33, wherein the animal expresses the exogenous IL33.

27. The animal of claim 26, wherein the exogenous IL33 polypeptide comprises an amino acid sequence that is at least 90%, 95%, or 99% identical to SEQ ID NO: 8, SEQ ID NO: 9, SEQ ID NO: 10, SEQ ID NO: 11, or SEQ ID NO: 12.

28. The animal of claim 26 or 27, wherein the nucleotide sequence is operably linked to an endogenous IL33 regulatory element of the animal.

29. The animal of any one claims 26-28, wherein the nucleotide sequence is integrated to an endogenous IL33 gene locus of the animal.

30. The animal of any one of claims 26-29, wherein the animal in its genome comprises from 5′ to 3″: a mouse 5′ UTR, a sequence encoding the exogenous IL33 polypeptide, and a mouse 3′ UTR.

31. A method of making a genetically-modified mouse cell that expresses a chimeric IL33, the method comprising:
a) replacing at an endogenous mouse IL33 gene locus, a nucleotide sequence encoding a region of mouse IL33 with a nucleotide sequence encoding a corresponding region of human IL33, thereby generating a genetically-modified mouse cell that includes a nucleotide sequence that encodes the chimeric IL33, wherein the mouse cell expresses the chimeric IL33.

32. The method of claim 31, wherein the nucleotide sequence encoding the chimeric IL33 is operably linked to an endogenous IL33 regulatory region, e.g., promoter.

33. The animal of any one of claims 1-21 and 26-30, wherein the animal further comprises a sequence encoding an additional human or chimeric protein.

34. The animal of claim 33, wherein the additional human or chimeric protein is programmed cell death protein 1 (PD-1), cytotoxic T-lymphocyte-associated protein 4 (CTLA-4), Lymphocyte Activating 3 (LAG-3), IL15 receptor, B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

35. The animal of claim 33, wherein the additional human or chimeric protein is PD-1.

36. The method of any one of claims 22-25, 31, and 32, wherein the animal or mouse further comprises a sequence encoding an additional human or chimeric protein.

37. The method of claim 36, wherein the additional human or chimeric protein is PD-1, CTLA-4, LAG-3, IL15 receptor, BTLA, PD-L1, CD3, CD27, CD28, CD47, CD137, CD154, TGIT, TIM-3, GITR, SIRPα or OX40.

38. The method of claim 36, wherein the additional human or chimeric protein is PD-1.

39. A method of determining effectiveness of an anti-IL33 antibody for treating an allergic disorder, comprising:
a) administering the anti-IL33 antibody to the animal of any one of claims 1-21 and 26-30, wherein the animal has the allergic disorder; and
b) determining effects of the anti-IL33 antibody in treating the allergic disorder.

40. The method of claim 39, wherein the allergic disorder is asthma.

41. The method of claim 40, wherein the animal is a mouse and the asthma is induced by treating the mouse with ovalbumin and aluminum hydroxide.

42. The method of claim 40 or 41, wherein the effects are evaluated by serum IgE levels;
pathological lung histology features; number of leukocytes (CD45+ cells), eosinophils (Eos), or neutrophils in bronchoalveolar lavage fluid (BALF); or percentages of eosinophils or neutrophils cells in CD45+ cells in bronchoalveolar lavage fluid (BALF).

43. The method of claim 39, wherein the allergic disorder is hay fever.

44. A method of determining effectiveness of an anti-IL33 antibody for reducing an inflammation, comprising:
a) administering the anti-IL33 antibody to the animal of any one of claims 1-21 and 26-30, wherein the animal has the inflammation; and
b) determining effects of the anti-IL33 antibody for reducing the inflammation

45. A method of determining effectiveness of an anti-IL33 antibody for treating an autoimmune disorder, comprising:
a) administering the anti-IL33 antibody to the animal of any one of claims 1-21 and wherein the animal has the autoimmune disorder; and
b) determining effects of the anti-IL33 antibody for treating the auto-immune disease.

46. A method of determining effectiveness of an anti-IL33 antibody for treating a cancer, comprising:
a) administering the anti-IL33 antibody to the animal of any one of claims 1-21 and 26-30, wherein the animal has the cancer; and
b) determining inhibitory effects of the anti-IL33 antibody for treating the cancer.

47. The method of claim 46, wherein the cancer is a tumor, and determining the inhibitory effects of the treatment involves measuring the tumor volume in the animal.

48. The method of claim 46 or 47, when the cancer is breast cancer, non-small-cell lung cancer (NSCLC), colorectal cancer, gastric cancer, hepatocellular carcinoma (HCC), hepatobiliary cancer, pancreatic cancer, lung cancer, prostate cancer, kidney cancer, ovarian cancer, uterine cancer, endometrial cancer, cervical cancer, head and neck cancer, brain cancer, glioma, gingivitis and salivary cancer, skin cancer, squamous cell carcinoma, blood cancer, lymphoma, or bone cancer.

49. A method of determining toxicity of an anti-IL33 antibody, the method comprising
a) administering the anti-IL33 antibody to the animal of any one of claims 1-21 and 26-30; and
b) determining weight change of the animal.

50. The method of claim 49, the method further comprising performing a blood test (e.g., determining red blood cell count).

51. A protein comprising an amino acid sequence, wherein the amino acid sequence is one of the following:
an amino acid sequence set forth in SEQ ID NOS: 8-12;
an amino acid sequence that is at least 90% identical to SEQ ID NOS: 8-12;
(c) an amino acid sequence that is at least 91%, 92%, 93%, 94%, 95%, 96%, 97%, 98%, or 99% identical to SEQ ID NO: 8-12;
(d) an amino acid sequence that is different from the amino acid sequence set forth in SEQ ID NOS: 8-12 by no more than 10, 9, 8, 7 , 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NOS: 8-12.

52. A nucleic acid comprising a nucleotide sequence, wherein the nucleotide sequence is one of the following:
(a) a sequence that encodes the protein of claim 51;
(b) SEQ ID NO: 7;
(c) a sequence that is at least 90% identical to SEQ ID NO: 7; and
(d) a sequence that is at least 91%, 92%, 93%, 94%, 95%, 96%, 97%, 98%, or 99% identical to SEQ ID NO: 7.

53. A cell comprising the protein of claim 51 and/or the nucleic acid of claim 52.

54. An animal comprising the protein of claim 51 and/or the nucleic acid of claim 52.


SEQUENCE LISTING (30 sequences)

SEQ ID NO: 1 - DNA, 2538 bp, Mouse
aagaggaactgcagctgcagaagggagaaatcacggcagaatcatcgagaaacctgaaaaatgagacctagaatgaagtattccaactccaagatttccccggcaaagttcagcagcaccgcaggcgaagccctggtcccgccttgcaaaataagaagatcccaacagaagaccaaagaattctgccatgtctactgcatgagactccgttctggcctcaccataagaaaggagactagttattttaggaaagaacccacgaaaagatattcactaaaatcgggtaccaagcatgaagagaacttctctgcctatccacgggattctaggaagagatccttgcttggcagtatccaagcatttgctgcgtctgttgacacattgagcatccaaggaacttcacttttaacacagtctcctgcctccctgagtacatacaatgaccaatctgttagttttgttttggagaatggatgttatgtgatcaatgttgacgactctggaaaagaccaagagcaagaccaggtgctactacgctactatgagtctccctgtcctgcaagtcaatcaggcgacggtgtggatgggaagaagctgatggtgaacatgagtcccatcaaagacacagacatctggctgcatgccaacgacaaggactactccgtggagcttcaaaggggtgacgtctcgcctccggaacaggccttcttcgtccttcacaaaaagtcctcggactttgtttcatttgaatgcaagaatcttcctggcacttacataggagtaaaagataaccagctggctctagtggaggagaaagatgagagctgcaacaatattatgtttaagctctcgaaaatctaatgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccacaaa

SEQ ID NO: 2 - protein, 266 aa, Mouse
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 3 - DNA, 2718 bp, human
agtctacagactcctccgaacacagagctgcagctcttcagggaagaaatcaaaacaagatcacaagaatactgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactactaaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgtaaaaaaaaaaaaa

SEQ ID NO: 4 - DNA, 24 bp, Artificial Sequence
  primer
gctccaatttcccacaacattagt

SEQ ID NO: 5 - DNA, 4218 bp, Artificial Sequence
  5!/ arm
aagggcacgcatatatcctttttacaatcagaaaaatatttacctgaaagaaagaaacaaaagaacaaaacaaaacaacaaaacaaacaaacaaaaaaccaactttctccagcaaaggcatcactgagcatctgttttaaaagttacatttaaaagccagtaattaagtatatctctgaaaggacgggtctgtggggaatctgtctcctgggaacagctacacaccctcacattccattcatatcgctccatttgcataactttactcatcagcttctgttaccaaagttgtttaatctgagctaccatataaatagaactgaagcgcgactgtgttagctctccaccggggctcactgcaggaaagtacagcattcaagaccagctatttcctgtctgtattgaggtgagtgccaggtcaatctctctgtttctgaggggaaccaatagtagcatctatcagctctcaaatgattctgatataaagagagaaaagacttgttactggagaaagtggcttgtttatcagaatgggatttgaaaccagttctgggttatacttttagttctgcaattgtgataaagatgctgacagtcttggtcgatgtatctgtccttaatgctcactccttagtttcttatctgttcaaccacgcaggctagtagtcctttagagcaaagttcttctacatgagtttgctcaagctcgtccaagggaggacgaaagggaagggaaggttaggagatcaaataattaactgaagatagagaaaggcaaaggcaggcagggcgtctcagtgaggggctgctggtattgttattatgttaacatgcatgaactctaagtctccaagaagagaggctatcgactgcaacacacaggtttatttaaaagacaattttaatgactagaaaaacattgtttctaggacacatgcaccctctggtgaaagagaaaacagaaatcagcactgtttcaggaagcagctcagaagctcagcactagcagtgagcaagggacctcaattcctcctggtacaggcaggctattatttaaactgtttcctgcccaactgaaaatctgacctttatgtggttagctaatcctattaccccacacagaaggcttaaaaattaactattctgtaaaagctataactctgctttaaaggaaattatgtattctgagccagaaaggatgactgaccattgtggcttagtagtatggattcaagttaccctaaatgatacattcctaagtgaaagccattacattaagggtttcaggcaaagaataaaggccaccatacaccaatgtattcaataactgcattagtcataacacgttcgacacacagaagacacaagaaggaggaggtctctcttgtaggttttgaatgcttgcagcttcttagctttgtgctatgcataaacagcccattctgagagtatcaaatcagatgccattattaaaagaaatggctgtttacacaaaaagcctaagacagccagagataaagtttggctcttgatcattgaccaatgatcaagacaatgacaatattttactcagtgaacgagaagagtacgtgggattatttaacccagccgcagcttcttcagggaactctgccttatgttagttaacatcttccgaaatttattattactatttcaagaatattaatatttcaagaagctggccatttgctttgagtataagaagggctgagtgtttgaaataaaataccaagttctaatccacccgactcccaccccccaagccgcaccccgcacctcctcgaagttgtcaactgatgcagttgttggatgtattggctggcatatggttgcctttgttctttgcctgaaagccttcatgcgactgctttccgcttaggagtgcattgtttagggaaacttgaatcaatgctactaagccatgatcagctatcatcgctggctcagaataaatggttcccttcaaagcagagttgtaacttttatagagtagttaatttttaatccttttgaacaaaagagagagagagagaggatgataatggagggagagagaagagataacagttttagtttcattgggggggtggtacaggataagagataagttctactttcattcttctacacatggatatccagctctcccagtttaaaaaaatttttaaggctatatttttctgatgtgtttttgatgccactagcaagaattagacggttctattggcctgagtatgacataattatattttgatttttaaaaaatttaagaaaaaatagacaatgtgttaaggttgaaacagtgtggggaagataacaagatttaaaactcaaatgtaaaagaagtagataaaattagacagaagagggataaaaatgtttttaaaaatcaaaataataagtaatagttttaaatgagaggaaaataaaaagagagacggaagcacctttccgggtctgtgagcagagggcagagggctagagcttgctcccactaagctggctagaatagcgaggccagttgagagcacccatcctcagcctcagggctgtgtttactctagactgtaaaagcgtagatgctttcttcctttgcacttatggggccatggcatcccccgacgatgtctacattccttaggcttgggcaggttatcattcactgaggctctctggagtccgcgactgtggcaaagtccacctagttcacctcagaatgaagggagcaaacggctctcctagttatgtccagatggaagagcaatcagcctgtcctggtttataagtaatcttcagctacctgcttggaggcctcagtcatccccaatcccaggagctgagtagtcctcgccaccagagggcgggagcactgatcactgtgtcttccttcctcaactgagcaggctccccgtaattgaaacttctcattggttcgttcatccgaaccaatcccttccaggattgcctcatagccggtcactatggcagcgttcccaggcatcctgatttggcacctgtctgtattttctgatcttcagagttgctgctggtcacaacacttttaaactaccggagagtgacggtgtggcgtctttactctaggcagcagtggacaggaactcgcccttagacctaatttctgccctggcttgtgagaactgtactgttaaccatgggtttctcccgctgcgctgcgagcagcgcaattctattcttctggtagcctaaacccagatgaacttgtgattgttgctccctccctcctaatccttcccagtccctctccccatcccccaccaatgcctggaaccgggactggagctctggactgcttcctacttcgtgttatctttttgtttctccatgagttttaattgccctgttgtttttgttgttgttttggttttccccctttatgcccagtgatttctccactagatctccttgggctttggtttgtcctttgttaccctggtctttgaacttgtgagaaaatcggcaaccttactccatttattcttgaggaaccatgcttaatcttgatcactgcctgccattactctttttaaacattcagctggtgccaaatctgacccgaggaaaccctccacggtggctcccacatgcttttgacacaaactcatttagttttttaatagctggacgttttctggcataacgtgttttatggtcattattactttcctgaccacgaatctggagtcaaagttccttccagtggagaaaatatttagaaaataaaatgtgtacacacactgcttgtttaatactgactcaaatctaacatcgcagggcatttgccttggcttctgagttacgtatttatatttcttacccccctacagtgagaaatcatacaagatcagtatcgctattttttttttttttttgctatatactacaacaaaacagtactgagatttcaacacaaacatggcattaacactaagactactcagcctcagatttctctgtgctttcattactttattctttgattctatggctagattctagcctaaatgtagagtcagaatactatgctgaattttattctcccccctcccccctgtatggctatcagtttcatggtgagatagccaaggttgcttctgatgacttcaggtccatatagttggattaatgttatatttcaatcccacagaaacctgaaaa

SEQ ID NO: 6 - DNA, 4081 bp, Artificial Sequence
  3!/ arm
tgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccacaaagcgctccaacgttccgagttcttaaatgaaaatgagtataccccagtgcatcattgttgacaagtgaggccggtggctgtttgacaacttagggtgttaagttagactttccggaacacggatgtgtctgtttcaccttcatccccccagctcctccactctctcaaataggagactttcgcaccaaaccaaacatacgcccccatggtcctacatcaaatattaacctgaaattcccgatcccttatttcttttctgttttgctctcctttagaacatattttcagagctgaggcgacattttgagcaagatgaggaccagggttcagaccccaagctcccatatagaagccaagagtggctgtaagccccagggctaaggaggctgatcctgggatgctgctggccctgccagcctaaccaagctggtgagctctgggttctgagactctgtctcagatagaggaagagttctgacattgatctctggtttccacacacgtgcatatgtgcacaccacacacacacacacacacacacacacacacacacacacatacactttccacattactacaatcagtgttattgattaaaatattcaaacatattggcattttgtcatacttaacatgtgcttcatggctaataataaatattactgggaataatctgtccattcaattatatcagaaatgataattttatctgatatctgaaaatttagtcatgtaatgtaatcttgtttatgttctttaggtctttcttgatgtatttggaggatttttgaaaatttttatttaaaaaaatttttttaaagatttatttatttattatatgtaagtacactgtagctgtcctcagatactccaggagagagcatcagatttcattatggatcgttgtgagccaccatgtggttgctgagatttgaactcggaaccttcggaagagcagttggcactctgagccatctcaccagcccaatttaaaatttttttatacaaagttttttgatagtattctcttccctcccctaattcctttcagattttctccacatccactttctgggtttgttttgttttgttattttggggttgtttttttgggggggtgggggggtgagtgtggaggaagtggctctctctttctctaaagaaaaaaaaacataaaatcaaagcagacaaactaaaaaagagaaggtagataagacatcaagacgaaacaaaacacacccacaaaaccatggtgtccgtcttgtgttgctcagctacccctggcacgacgctctgcagtcttgtgcgtgctctcacagtctctgtgggatcctgtgtttatcagcacttcctgtgtctggaatacagtttccttgaggtcctggggacactgcttcctcttcttgatggatccctgattctcaatgggagaggttttgtgaagatatctcacttagggccgagtgctccaaactctccccctctctgtaccttgtccattgtggatctctttgttaattcacatctaccacaagaagaagcttcccaaagaggggttaagtgacactctgctctgtgtgtacagtcatatgtcattaagactcattctgctagccaggtggtggcggtgcacgcctttaatcccagcacttgggagggggatttccgagttcgaggccagcctggtctacaaagtgagttccaggacagccagggctacacagagaaaccctgtcaaaaaaccaaaaacaaacaaaaaaaacaaaaaaaacaccaagaatcattctgctcctttaacagaaaaatactagattttcccgtaggcctctgacctctctagccctaggttcttggccaattcagcagggacaggtatgggctccatctcatgaacccagtttttaaaaatggttggttattctgtaacattgttccacaggtctatcacgcaggcaggtagatcgctgggtaagacttgataattgcttttctcccccagtagactgcaaagtaccttgagaactctgagcgcttgtcagtagggtgaagcttttggttgggcacaggctcaatttcttatgttcagtgacatagagaagctgctgtcttcagtagtagggtcttaccatcgggttacagagagtaagcaactgccttaagcaacagtttgtgctgcggggaggttagcatatggaatcactttggccaacaactcaccaagatttaactcacagaagatgccttgttggggcattgtctcctacattatatggtgactccatttaaattccttttctatatagtacatcttttagaaagcttccgtggtattagttgtgcatctggctttttaaaagttctttattgtgagttatctgtccccgtattctctcctttcccttccc

SEQ ID NO: 7 - DNA, 2550 bp, Artificial Sequence
  The modified humanized mouse IL33 mRNA sequence
aagaggaactgcagctgcagaagggagaaatcacggcagaatcatcgagaaacctgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagtgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccacaaa

SEQ ID NO: 8 - protein, 270 aa, Artificial Sequence
  expressed protein of the modified humanized mouse IL33
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 9 - protein, 228 aa, Artificial Sequence
  expressed protein of the modified humanized mouse IL33
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 10 - protein, 144 aa, Artificial Sequence
  expressed protein of the modified humanized mouse IL33
MKPKMKYSTNKISTAKWKNTASKALCFKLGNKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 11 - protein, 264 aa, Artificial Sequence
  expressed protein of the modified humanized mouse IL33
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 12 - protein, 228 aa, Artificial Sequence
  expressed protein of the modified humanized mouse IL33
MKPKMKYSTNKISTAKWKNTASKALCFKLGKSQQKAKEVCPMYFMKLRSGLMIKKEACYFRRETTKRPSLKTGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 13 - DNA, 71 bp, Artificial Sequence
  The connection between the 5′ end of the Neo cassette
cattctgagcctgcttaagggagagtcagtcgacggtatcgataagcttgatatcgaattccgaagttcct

SEQ ID NO: 14 - DNA, 70 bp, Artificial Sequence
  The connection between the 3!/ end of the Neo cassette
tcctattctctagaaagtataggaacttcatcagtcaggtacataatggtggatcctaagcgtttccatg

SEQ ID NO: 15 - DNA, 20 bp, Artificial Sequence
  primer
gctcgactagagcttgcgga

SEQ ID NO: 16 - DNA, 25 bp, Artificial Sequence
  primer
agaggctcttacagggaaggggata

SEQ ID NO: 17 - DNA, 25 bp, Artificial Sequence
  primer
aggagaagcctagaaagagcccagt

SEQ ID NO: 18 - DNA, 25 bp, Artificial Sequence
  primer
tgcttgctgtgttcttccactttgc

SEQ ID NO: 19 - DNA, 25 bp, Artificial Sequence
  probe
tatagctggtcacgtggtagcctca

SEQ ID NO: 20 - DNA, 25 bp, Artificial Sequence
  probe
actgggctctttctaggcttctcct

SEQ ID NO: 21 - DNA, 25 bp, Artificial Sequence
  probe
aggctagcactcacccttactctcc

SEQ ID NO: 22 - DNA, 25 bp, Artificial Sequence
  probe
tagatcgagaggtgcacagtcaagc

SEQ ID NO: 23 - DNA, 23 bp, Artificial Sequence
  probe
ggatcggccattgaacaagatgg

SEQ ID NO: 24 - DNA, 23 bp, Artificial Sequence
  probe
cagaagaactcgtcaagaaggcg

SEQ ID NO: 25 - DNA, 26 bp, Artificial Sequence
  primer
aggttgcttctgatgacttcaggtcc

SEQ ID NO: 26 - DNA, 25 bp, Artificial Sequence
  primer
aggtcgcccgtcttcatgttgaaat

SEQ ID NO: 27 - DNA, 25 bp, Artificial Sequence
  primer
ttatctcagctattcctgcctggtg

SEQ ID NO: 28 - DNA, 25 bp, Artificial Sequence
  primer
ccattctgagcctgcttaagggaga

SEQ ID NO: 29 - DNA, 25 bp, Artificial Sequence
  primer
atcttggcacatggaaacgcttagg

SEQ ID NO: 30 - DNA, 25 bp, Artificial Sequence
  primer
gacaagcgttagtaggcacatatac